SEQUENCE LISTING (42 sequences)

SEQ ID NO: 1 - protein, 500 aa, Human papillomavirus type 6
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKQKPDPYKNLSFWEVNLKEKFSSELDQYPLGRKFLLQSGYRGRSSIRTGVKRPAVSKASAAPKRKRAKTKR

SEQ ID NO: 2 - protein, 496 aa, Artificial Sequence
  HPV6 L1
MDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMAGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVVAYIHTMNPSVLEDWNFGLSPPPNGTLEDTYRYVQSQAITCQKPTPEKQKPDPYKNLSFWEVNLKEKFSSELDQYPLGRKFLLQSGYRGRSSIRTGVKRPAVSKASAAPKRKRAKTKR

SEQ ID NO: 3 - protein, 501 aa, Human papillomavirus type 11
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 4 - protein, 498 aa, Artificial Sequence
  HPV11 L1
MSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 5 - protein, 531 aa, Human papillomavirus type 16
MEVTFIYILVITCYENDVNVYHIFFQMSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 6 - protein, 502 aa, Artificial Sequence
  HPV16 L1
MLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGDNVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADIMTYIHSMNSTILEDWNFGLQPPPGGTLEDTYRFVTSQAIACQKHTPPAPKEDPLKKYTFWEVNLKEKFSADLDQFPLGRKFLLQAGLEAKPKFTLGKRKATPTTSSTSTTAKRKKRKL

SEQ ID NO: 7 - protein, 507 aa, Human papillomavirus type 18
MALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 8 - protein, 504 aa, Artificial Sequence
  HPV18 L1
MRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAIACQKDAAPAENKDPYDKLKFWNVDLKEKFSLDLDQYPLGRKFLVQAGLRRKPTIGPRKRSAPSATTASKPAKRVRVRARK

SEQ ID NO: 9 - protein, 504 aa, Human papillomavirus type 31
MSLWRPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKTAPQKPKEDPFKDYVFWEVNLKEKFSADLDQFPLGRKFLLQAGYRARPKFKAGKRSAPSASTTTPAKRKKTKK

SEQ ID NO: 10 - protein, 504 aa, Artificial Sequence; identical to SEQ ID NO: 9
  HPV31 L1

SEQ ID NO: 11 - protein, 499 aa, Human papillomavirus type 33
MSVWRPSEATVYLPPVPVSKVVSTDEYVSRTSIYYYAGSSRLLAVGHPYFSIKNPTNAKKLLVPKVSGLQYRVFRVRLPDPNKFGFPDTSFYNPDTQRLVWACVGLEIGRGQPLGVGISGHPLLNKFDDTETGNKYPGQPGADNRECLSMDYKQTQLCLLGCKPPTGEHWGKGVACTNAAPANDCPPLELINTIIEDGDMVDTGFGCMDFKTLQANKSDVPIDICGSTCKYPDYLKMTSEPYGDSLFFFLRREQMFVRHFFNRAGTLGEAVPDDLYIKGSGTTASIQSSAFFPTPSGSMVTSESQLFNKPYWLQRAQGHNNGICWGNQVFVTVVDTTRSTNMTLCTQVTSDSTYKNENFKEYIRHVEEYDLQFVFQLCKVTLTAEVMTYIHAMNPDILEDWQFGLTPPPSASLQDTYRFVTSQAITCQKTVPPKEKEDPLGKYTFWEVDLKEKFSADLDQFPLGRKFLLQAGLKAKPKLKRAAPTSTRTSSAKRKKVKK

SEQ ID NO: 12 - protein, 491 aa, Artificial Sequence
  HPV33 L1
MTVYLPPVPVSKVVSTDEYVSRTSIYYYAGSSRLLAVGHPYFSIKNPTNAKKLLVPKVSGLQYRVFRVRLPDPNKFGFPDTSFYNPDTQRLVWACVGLEIGRGQPLGVGISGHPLLNKFDDTETGNKYPGQPGADNRECLSMDYKQTQLCLLGCKPPTGEHWGKGVACTNAAPANDCPPLELINTIIEDGDMVDTGFGCMDFKTLQANKSDVPIDICGSTCKYPDYLKMTSEPYGDSLFFFLRREQMFVRHFFNRAGTLGEAVPDDLYIKGSGTTASIQSSAFFPTPSGSMVTSESQLFNKPYWLQRAQGHNNGICWGNQVFVTVVDTTRSTNMTLCTQVTSDSTYKNENFKEYIRHVEEYDLQFVFQLCKVTLTAEVMTYIHAMNPDILEDWQFGLTPPPSASLQDTYRFVTSQAITCQKTVPPKEKEDPLGKYTFWEVDLKEKFSADLDQFPLGRKFLLQAGLKAKPKLKRAAPTSTRTSSAKRKKVKK

SEQ ID NO: 13 - protein, 513 aa, Human papillomavirus type 45
MALWRPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 14 - protein, 513 aa, Artificial Sequence; identical to SEQ ID NO: 13
  HPV45 L1

SEQ ID NO: 15 - protein, 529 aa, Human papillomavirus type 52
MVQILFYILVIFYYVAGVNVFHIFLQMSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 16 - protein, 490 aa, Artificial Sequence
  HPV52 L1
MPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 17 - protein, 524 aa, Human papillomavirus type 58
MVLILCCTLAILFCVADVNVFHIFLQMSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 18 - protein, 490 aa, Artificial Sequence
  HPV58 L1
MTVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 19 - protein, 500 aa, Artificial Sequence
  HPV6 L1
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMAGCAPPLGEHWGKGKQCTNTPVQAGDCPPLELITSVIQDGDMVDTGFGAMNFADLQTNRSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKQKPDPYKNLSFWEVNLKEKFSSELDQYPLGRKFLLQSGYRGRSSIRTGVKRPAVSKASAAPKRKRAKTKR

SEQ ID NO: 20 - protein, 502 aa, Artificial Sequence
  HPV11 L1
MWRPMSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 21 - protein, 505 aa, Artificial Sequence
  HPV16 L1
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 22 - protein, 507 aa, Artificial Sequence
  HPV18 L1
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGICWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 23 - protein, 504 aa, Artificial Sequence
  HPV31 L1
MSLWRPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKSAPQKPKEDPFKDYVFWEVNLKEKFSADLDQFPLGRKFLLQAGYRARPKFKAGKRSAPSASTTTPAKRKKTKK

SEQ ID NO: 24 - protein, 499 aa, Artificial Sequence; identical to SEQ ID NO: 11
  HPV33 L1

SEQ ID NO: 25 - protein, 510 aa, Artificial Sequence
  HPV45 L1
MALWRPSDSTVYLPPPSVARVVNTDDYVSRTSIFYHAGSSRLLTVGNPYFRVVPSGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSITTSDSQLFNKPYWLHKAQGHNNGICWHNQLFVTVVDTTRSTNLTLCASTQNPVPNTYDPTKFKHYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASRPAKRVRIRSKK

SEQ ID NO: 26 - protein, 503 aa, Artificial Sequence
  HPV52 L1
MSVWRPSEATVYLPPVPGSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 27 - protein, 498 aa, Artificial Sequence
  HPV58 L1
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYFNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSMVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 28 - protein, 471 aa, Artificial Sequence
  HPV16 L1
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQ

SEQ ID NO: 29 - protein, 472 aa, Artificial Sequence
  HPV18 L1
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACVGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPPSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQ

SEQ ID NO: 30 - protein, 503 aa, Human papillomavirus type 26
MALWRTSDSKVYLPPTPVSRVVNTDEYVTRTGIYYYAGSSRLLTLGHPYFSIPKTGQKAEIPKVSAYQYRVFRVHLPDPNKFGLPDPQLYNPDTERLVWACVGVEVGRGQPLGIGLSGHPLFNKLDDTENSHLATVNADTDNRDNVSVDNKQTQLCIIGCTPPLGEHWGIGTICKNTQTQRGDCPPLELISSIIEDGDMIDTGFGAMDFTALQATKSDVPIDISQSTCKYPDYLKMSADTYGNSMFFFLRREQLFARHFYNKAGAVGDAIPTTLYIKGAESGREPPTSSIYSATPSGSMVTSDAQLFNKPYWLQRAQGHNNGICWGNQLFVTCVDTTRSTNLTISTLSAASASTPFKPSDYKQFIRHGEEYELQFIFQLCKITLTTDVMAYIHLMNASILEDWNFGLTLPPTASLEDAYRFIKNSATTCQRNAPPVPKEDPFQKFKFWDVDLKEKFSIDLDQFPLGRKFMLQAGIQRRPKLGTKRPLSSTSSSTKRKKRKLTK

SEQ ID NO: 31 - protein, 502 aa, Human papillomavirus type 35
MSLWRSNEATVYLPPVSVSKVVSTDEYVTRTNIYYHAGSSRLLAVGHPYYAIKKQDSNKIAVPKVSGLQYRVFRVKLPDPNKFGFPDTSFYDPASQRLVWACTGVEVGRGQPLGVGISGHPLLNKLDDTENSNKYVGNSGTDNRECISMDYKQTQLCLIGCRPPIGEHWGKGTPCNANQVKAGECPPLELLNTVLQDGDMVDTGFGAMDFTTLQANKSDVPLDICSSICKYPDYLKMVSEPYGDMLFFYLRREQMFVRHLFNRAGTVGETVPADLYIKGTTGTLPSTSYFPTPSGSMVTSDAQIFNKPYWLQRAQGHNNGICWSNQLFVTVVDTTRSTNMSVCSAVSTSDSTYKNDNFKEYLRHGEEYDLQFIFQLCKITLTADVMTYIHSMNPSILEDWNFGLTPPPSGTLEDTYRYVTSQAVTCQKPSAPKPKDDPLKNYTFWEVDLKEKFSADLDQFPLGRKFLLQAGLKARPNFRLGKRAAPASTSKKSSTKRRKVKS

SEQ ID NO: 32 - protein, 505 aa, Human papillomavirus type 39
MAMWRSSDSMVYLPPPSVAKVVNTDDYVTRTGIYYYAGSSRLLTVGHPYFKVGMNGGRKQDIPKVSAYQYRVFRVTLPDPNKFSIPDASLYNPETQRLVWACVGVEVGRGQPLGVGISGHPLYNRQDDTENSPFSSTTNKDSRDNVSVDYKQTQLCIIGCVPAIGEHWGKGKACKPNNVSTGDCPPLELVNTPIEDGDMIDTGYGAMDFGALQETKSEVPLDICQSICKYPDYLQMSADVYGDSMFFCLRREQLFARHFWNRGGMVGDAIPAQLYIKGTDIRANPGSSVYCPSPSGSMVTSDSQLFNKPYWLHKAQGHNNGICWHNQLFLTVVDTTRSTNFTLSTSIESSIPSTYDPSKFKEYTRHVEEYDLQFIFQLCTVTLTTDVMSYIHTMNSSILDNWNFAVAPPPSASLVDTYRYLQSAAITCQKDAPAPEKKDPYDGLKFWNVDLREKFSLELDQFPLGRKFLLQARVRRRPTIGPRKRPAASTSSSSATKHKRKRVSK

SEQ ID NO: 33 - protein, 504 aa, Human papillomavirus type 51
MALWRTNDSKVYLPPAPVSRIVNTEEYITRTGIYYYAGSSRLITLGHPYFPIPKTSTRAAIPKVSAFQYRVFRVQLPDPNKFGLPDPNLYNPDTDRLVWGCVGVEVGRGQPLGVGLSGHPLFNKYDDTENSRIANGNAQQDVRDNTSVDNKQTQLCIIGCAPPIGEHWGIGTTCKNTPVPPGDCPPLELVSSVIQDGDMIDTGFGAMDFAALQATKSDVPLDISQSVCKYPDYLKMSADTYGNSMFFHLRREQIFARHYYNKLGSVGEDIPNDYYIKGSGNGRDPIESYIYSATPSGSMITSDSQIFNKPYWLHRAQGHNNGICWNNQLFITCVDTTRSTNLTISTATAAVSPTFTPSNFKQYIRHGEEYELQFIFQLCKITLTTEVMAYLHTMDPTILEQWNFGLTLPPSASLEDAYRFVRNAATSCQKDTPPQAKPDPLAKYKFWDVDLKERFSLDLDQFALGRKFLLQVGVQRKPRPGLKRPASSASSSSSSSAKRKRVKK

SEQ ID NO: 34 - protein, 499 aa, Human papillomavirus type 53
MAVWRPSDSKVYLPPTPVSKVITTDAYVKRTTIFYHAGSSRLLTVGHPYYPISKSGKTDIPKVSAFQYRVFRVRLPDPNKFGLPDTNIFNPDQERLVWACVGLEIGRGQPLGVGVSGHPLFNRLDDTESSSIAIQDTAPDSRDNISVDPKQTQLCIIGCAPAIGEHWTKGTACRSTPTTAGDCPPLELINSPIEDGDMVDTGFGALNFKALQESKSDVPLDIVQSTCKYPDYLKMSADAYGDSMWFYLRREQLFTRHFFNRSGVIGEEIPNDLYIKGSNDRDPPPSSVYVATPSGSMITSEAQLFNKPYWLQRAQGHNNGICWNNQLFVTVVDTTRNTNMTLSATTQSMSTYNSKQIKQYVRHAEEYELQFVFQLCKISLSAEVMAYLHTMNSTLLEDWNIGLSPPVATSLEDKYRYVKSAAITCQKDQPPPEKQDPLSKYKFWEVNLQNSFSADLDQFPLGRKFLMQVGVRTEPPVSSKKRSAPTTSTSAPSSKRKRK

SEQ ID NO: 35 - protein, 499 aa, Human papillomavirus type 56
MATWRPSENKVYLPPTPVSKVVATDSYVKRTSIFYHAGSSRLLAVGHPYYSVTKDNTKTNIPKVSAYQYRVFRVRLPDPNKFGLPDTNIYNPDQERLVWACVGLEVGRGQPLGAGLSGHPLFNRLDDTESSNLANNNVIEDSRDNISVDGKQTQLCIVGCTPAMGEHWTKGAVCKSTQVTTGDCPPLALINTPIEDGDMIDTGFGAMDFKVLQESKAEVPLDIVQSTCKYPDYLKMSADAYGDSMWFYLRREQLFARHYFNRAGKVGETIPAELYLKGSNGREPPPSSVYVATPSGSMITSEAQLFNKPYWLQRAQGHNNGICWGNQLFVTVVDTTRSTNMTISTATEQLSKYDARKINQYLRHVEEYELQFVFQLCKITLSAEVMAYLHNMNANLLEDWNIGLSPPVATSLEDKYRYVRSTAITCQREQPPTEKQDPLAKYKFWDVNLQDSFSTDLDQFPLGRKFLMQLGTRSKPAVATSKKRSAPTSTSTPAKRKRR

SEQ ID NO: 36 - protein, 508 aa, Human papillomavirus type 59
MALWRSSDNKVYLPPPSVAKVVSTDDYVTRTSIFYHAGSSRLLTVGHPYFKVPKGGNGRQDVPKVSAYQYRVFRVNLPDPNKFGLPDNTVYDPNSQRLVWACVGVEIGRGQPLGVGLSGHPFYNKLDDTENSHVASAVDNKDTRDNVSVDYKQTQLCIIGCVPAIGEHWTKGTACKPNTVVQGDCPPLELINTPIEDGDMVDTGYGAMDFKLLQDTKSEVPLDICQSICKYPDYLQMSADAYGDSMFFCLRREQVFARHFWNRSGTMGDQIPESLYIKGTDIRATPGSYLYSPSPSGSVVTSDSQLFNKPYWLHKAQGLNNGICWHNQLFLTVVDTTRSTNLSVCASTTSSIPNVYTPTSFKEYARHVEEFDLQFIFQLCKITLTTEVMSYIHNMNTTILEDWNFGVTPPPTASLVDTYRFVQSAAVTCQKDTAPPVKQDPYDKLKFWTVDLKERFSADLDQFPLGRKFLLQLGARPKPTIGPRKRAAPAPTSTPSPKRVKRRKSSRK

SEQ ID NO: 37 - protein, 503 aa, Human papillomavirus type 66
MAMWRPSDNKVYLPPTPVSKVVATDTYVKRTSIFYHAGSSRLLAVGHPYYSVSKSGTKTNIPKVSAYQYRVFRVRLPDPNKFGLPDPSFYNPDQERLVWACVGLEVGRGQPLGAGLSGHPLFNRLDDTEVSNLAGNNVIEDSRDNISVDCKQTQLCIVGCAPALGEHWTKGAVCKSTPGNTGDCPPLALVNTPIEDGDMVDTGFGAMDFKLLQESKAEVPLDIVQSTCKYPDYLKMSADAYGDSMWFYLRREQLFARHYFNRAGNVGEAIPTDLYWKGGNGRDPPPSSVYVATPSGSMITSEAQLFNKPYWLQRAQGHNNGICWGNQVFVTVVDTTRSTNMTINAAKSTLTKYDAREINQYLRHVEEYELQFVFQLCKITLTAEVMAYLHNMNNTLLDDWNIGLSPPVATSLEDKYRYIKSTAITCQREQPPAEKQDPLAKYKFWEVNLQDSFSADLDQFPLGRKFLMQLGPRPPRPKASVSASKRRAAPTSSSSSPAKRKKR

SEQ ID NO: 38 - protein, 500 aa, Human papillomavirus type 67
MSVWRPSEATVYLPPVPVSKVVSTDEYVSRTSIYYYAGSSRLLAVGHPYFSIPNPSNTKKVLVPKVSGLQYRVFRVHLPDPNKFGFPDTSFYNPDTQRLVWACVGIEIGRGQPLGVGISGHPLLNKFDDTETNNKYPSQPGTDNRECLSMDAKQTQLCIIGCKPPTGEHWGKGTPCSGNSTGPGACPPLELMNTVIEDGDMIDTGFGCMDFKSLQANKSDVPLDICTSICKYPDYLGMASEAYGDSLFFFLRREQMFVRHLFNRAGKLGEDVPTDLYFKGSANTSALQTSAFFPTPSGSMVSSESQLFNKPYWLQRAQGHNNGICWGNQIFVTVVDTTRSTNMTLCSEEKSEATYKNENFKEYLRHVEEYDLQFIFQLCKISLTANVMQYIHTMNPDILEDWQFGLTPPPSGNLQDTYRFVTSQAITCQKTATPTAKEDPLKKYSFWEINLKEKFSADLDQFPLGRKFLLQAGFTAKPKLKRSSPSSSSSSSAKRKKVKR

SEQ ID NO: 39 - protein, 505 aa, Human papillomavirus type 68
MALWRSSDNMVYLPPPSVAKVVNTDDYVTRTGIYYYAGTSRLLTVGHPYFKVPMSGGRKQDIPKVSAYQYRVFRVSLPDPNKFSLPESTLYNPDTQRLVWACVGVEIGRGQPLGVGLSGHPLYNRLDDTENSPFSSNKNPKDSRDNVSVDYKQTQLCIIGCVPAIGEHWAKGKSCKPSNVQPGDCPPLELVNTPIQDGDMIDTGYGAMDFSTLQETKSEVPLDICQSVCKYPDYLQMSADVYGDSMFFCLRRDQLFARHFWNRGGMVGDTIPTELYIKGTDIRDSPSSYVYAPSPSGSMVSSDSQLFNKPNWLHKAQGHNNGICWHNQLFLTVVDTTRSTNFTLSTTTESAVPNIYDPNKFKEYVRHVEEYDLQFIFQLCTITLSTDVMSYIHTMNPAILDDWNFGVAPPPSASLVDTYRYLQSAAITCQKDAPAPTKKDPYDGLNFWNVNLKEKFSSELDQFPLGRKFLLQAGVRRRPTIGPRKRPATAPTASTSKHKRKRVSK

SEQ ID NO: 40 - protein, 506 aa, Human papillomavirus type 70
MALWRSSDNTVYLPPPSVAKVVNTDDYVTRTGIYYYAGSSRLLTVGHPYFKVPVNGGRKQEIPKVSAYQYRVFRVSLPDPNKFGLPDPSLYNPDTQRLVWACIGVEIGRGQPLGVGVSGHPLYNRLDDTENSHFSSAVNTQDSRDNVSVDYKQTQLCIIGCVPAMGEHWAKGKACKSTTVQQGDCPPLELVNTAIEDGDMIDTGYGAMDFRTLQETKSEVPLDICQSVCKYPDYLQMSADVYGDSMFFCLRKEQLFARHFWNRGGMVGDTIPSELYIKGTDIRDRPGTHVYSPSPSGSMVSSDSQLFNKPYWLHKAQGHNNGICWHNQLFITVVDTTRSTNFTLSACTETAIPAVYSPTKFKEYTRHVEEYDLQFIFQLCTITLTADVMAYIHTMNPAILDNWNIGVTPPPSASLVDTYRYLQSAAIACQKDAPAPEKKDPYDDLKFWNVDLKEKFSTELDQFPLGRKFLLQVGARRRPTIGPRKRPASAKSSSSASKHKRKRVSK

SEQ ID NO: 41 - protein, 503 aa, Human papillomavirus type 73
MWRPTDAKVYLPPVSVSKVVSTDEYVTRTNIYYYAGSTRLLAVGHPYFPIKDSQKRKTIVPKVSGLQYRVFRLRLPDPNKFGFPDASFYNPDKERLVWACSGVEVGRGQPLGIGTSGNPFMNKLDDTENAPKYIAGQNTDGRECMSVDYKQTQLCILGCRPPLGEHWGPGTPCTSQTVNTGDCPPLELKNTPIQDGDMIDVGFGAMDFKALQANKSDVPIDISNTTCKYPDYLGMAADPYGDSMWFYLRREQMFVRHLFNRAGDTGDKIPDDLMIKGTGNTATPSSCVFYPTPSGSMVSSDAQLFNKPYWLQKAQGQNNGICWHNQLFLTVVDTTRSTNFSVCVGTQASSSTTTYANSNFKEYLRHAEEFDLQFVFQLCKISLTTEVMTYIHSMNSTILEEWNFGLTPPPSGTLEETYRYVTSQAISCQRPQPPKETEDPYAKLSFWDVDLKEKFSAELDQFPLGRKFLLQLGMRARPKLQASKRSASATTSATPKKKRAKRI

SEQ ID NO: 42 - protein, 503 aa, Human papillomavirus type 82
MALWRTNDSKVYLPPAPVSRIVNTEEYITRTGIYYYAGSSRLITLGHPYFSIPKTNTRAEIPKVSAFQYRVFRVQLPDPNKFGLPDPNLFNPDTDRLVWGCVGVEVGRGQPLGVGLSGHPLFNKYDDTENSRFANGNDQQDVRDNISVDNKQTQLCIIGCAPPIGEHWATGTTCKNVPVPQGDCPPLELVSTVIEDGDMVDTGFGAMDFANLQATKSDVPLDIAQSVCKYPDYLKMSADTYGNSMFFHLRREQIFARHYYNKAGVVGDAIPDKAYIKGTGAGRDPISSYIYSATPSGSMITSDSQIFNKPYWLHRAQGHNNGICWNNQLFITCVDTTKSTNLTISTAVTPSVAQTFTPANFKQYIRHGEEYELQFIFQLCKITLTTEIMAYLHTMDSTILEQWNFGLTLPPSASLEDAYRFVKNAATSCHKDSPPQAKEDPLAKYKFWNVDLKERFSLDLDQFALGRKFLLQIGAQRKPRPGLKRPAPSSSASSSAKRKRVKK

(19) United States
(12) Patent Application Publication
(10) Pub. No.: US 2024/0131140 A1
(43) Pub. Date: Apr. 25, 2024

(54) HPV VACCINE MANUFACTURE

(71) Applicants: GLAXOSMITHKLINE BIOLOGICALS SA, Rixensart (BE); XIAMEN INNOVAX BIOTECH CO., LTD., Xiamen (CN)

(72) Inventors: Babak BAYAT, Rixensart (BE); Véronique HENDERICKX, Rixensart (BE); Bram VUYLSTEKE, Rixensart (BE)

(73) Assignees: GLAXOSMITHKLINE BIOLOGICALS SA, Rixensart (BE); XIAMEN INNOVAX BIOTECH CO., LTD., Xiamen (CN)

(21) Appl. No.: 18/276,578

(22) PCT Filed: Feb. 9, 2022
(86) PCT No.: PCT/EP2022/053143

§ 371 Date: Aug. 9, 2023

(30) Foreign Application Priority Data
Feb. 11, 2021 (EP) 21156664.1

(51) Int. Cl. A61K 39/12

(52) U.S. Cl. CPC A61K 39/12; A61K 2039/55505; C12N 2710/20034

(57) ABSTRACT

Described are methods for the preparation of a HPV vaccine composition by, for example (i) adsorbing one or more HPV antigen(s) on a metallic salt and, then (ii) adding a non adsorbed glycolipid based TLR4 ligand to the mixture obtained in (i). Resulting HPV vaccine compositions and uses thereof are also described.

### FIELD OF THE INVENTION

The present invention relates to the field of human vaccines. More particularly, the present invention relates to a HPV vaccine manufacturing process, and to resulting HPV vaccine compositions.

### BACKGROUND

Papillomaviruses are small, highly species specific, DNA tumor viruses. Human papillomaviruses are DNA viruses that infect basal epithelial (skin or mucosal) cells. Over 100 individual human papillomaviruses (HPV) types have been described. HPVs are generally specific either for the squamous epithelium of the skin or mucosal surfaces and usually cause benign tumors (warts) that persist for several months or years.

Persistent infection with an oncogenic human papillomavirus (HPV) type causes cervical cancer, the second most common cause of cancer deaths among women worldwide. There is international consensus that “high-risk” HPV types, including types 16, 18, 26, 31, 33, 35, 39, 45, 51, 52, 53, 56, 58, 59, 66, 67, 68, 70, 73 and 82 can lead to cervical cancer and are associated with other mucosal anogenital and head and neck cancers (IARC monograph 2018 https://monographs.iarc.fr/wp-content/uploads/2018/06/mono100B-11.pdf, Martinez-Portilla et al., 2019¹; Arbyn M et al., 2014²).

Infections with other types, termed “low-risk,” including types 6, 11, can cause benign or low-grade cervical tissue changes and genital warts (condyloma acuminata), which include growths on the cervix, vagina, vulva and anus in women and the penis, scrotum or anus in men. They also cause epithelial growths over the vocal cords of children and adults (juvenile respiratory papillomatosis or recurrent respiratory papillomatosis) that require surgical intervention (Bosch et al., 2013³).

Several prophylactic HPV vaccines are licensed so far. They all use virus-like particles (VLPs) comprised of recombinant L1 capsid proteins of individual HPV types.

The CERVARIX HPV vaccine contains HPV-16 and -18 VLPs produced in a Trichoplusia ni insect cell substrate using a baculovirus expression vector system and formulated with the immunostimulant 3-O-desacyl-4′-monophosphoryl lipid A (3D MPL, also known as MPL) and aluminum hydroxide salt.

GARDASIL-9 (a nine-valent HPV vaccine) is the evolution of GARDASIL (a four-valent HPV vaccine), and contains HPV-6, -11, -16, -18, -31, -33, -45, -52, and -58 VLPs produced in the yeast Saccharomyces cerevisiae and formulated with amorphous aluminum hydroxyphosphate sulphate salt. GARDASIL-9 contains VLPs from non-oncogenic types HPV-6 and -11, which are implicated in 75-90% of genital warts, and from oncogenic types HPV-16, -18, -31, -33, -45, -52 and -58 implicated in cervical, vulvar, vaginal, anal, oropharyngeal and other head and neck cancers.

As reviewed by Fox et al. 2017⁴ in “Current Status of Toll-Like Receptor 4 Ligand Vaccine Adjuvants”, TLR4 ligands demonstrate excellent adjuvant properties and TLR4-based adjuvant formulations have been included in several clinical trials; in particular 3D-MPL, GLA, SLA, RC-529 that can be adsorbed onto aluminum, and others such as OM-174 (E. coli derived triacetylated disaccharide diphosphoryl compound), PET lipid A (hexa-acylated monosaccharide monophosphoryl compound), ONO-4007 (monosaccharide triacyl structure with two of the chains terminating in benzene rings and a sulphate head group). Additionally, dLOS is another TLR4 ligand. It is prepared by alkaline hydrolysis of LPS which lacks O-antigen and is expressed by Escherichia coli rough strain. Furthermore, upon combination with aluminum, dLOS and alum are capable of synergizing their adjuvant effects to HPV L1 VLPs (Eun et. al., 2014⁶).

WO00/23105 discloses a process for the preparation of an adjuvanted HPV vaccines which consists of admixing HPV L1 VLPs and TLR4 ligands that were each pre-adsorbed onto a metallic salt.

There is a need for improved methods of preparing adjuvanted HPV vaccines.

### SUMMARY OF THE INVENTION

In one aspect, the present invention provides a method for the preparation of a HPV vaccine composition comprising the steps of (i) adsorbing one or more HPV antigen(s) on a metallic salt and, (ii) adding a non adsorbed glycolipid based TLR4 ligand to the mixture obtained in (i).

In another aspect, the invention provides a HPV vaccine composition obtained by the method according to the invention.

In one aspect, there is provided a HPV vaccine composition as described herein for use in therapy.

In one aspect, there is provided a HPV vaccine composition as described herein for preventing or treating a HPV infection or associated disease in a subject.

In one aspect, there is provided the use of a HPV vaccine composition as described herein, in the manufacture of a medicament for the prevention or treatment of HPV infection in a subject.

In one aspect, there is provided a method of preventing or treating an infection or disease comprising the administration of an effective amount of a HPV vaccine composition as described herein to a patient in need thereof.

In one aspect, there is provided a method of preventing or treating an infection or disease caused by HPV comprising the administration of an effective amount of a HPV vaccine composition as described herein to a patient in need thereof.

### BRIEF DESCRIPTION OF THE DRAWINGS

FIG. 1: Picture of 9V DP after one day (A) and 7 days (B). From left to right: in-line AlOOH (Ix); in-line no Al added; in-line AlPO₄; in-line AlOOH (Croda); in-line AlOOH (Ix) with aggregated MPL; in-line AlPO₄ with aggregated MPL; in-line AlOOH (Croda) with aggregated MPL; 9V DP with AMB MPL AlPO₄; 9V DP with AMB MPL AlOOH; 9V DP with AMB aggregated MPL AlPO₄.

FIG. 2: Graph representing the evolution of the average median particle size measured by SLS over 4 time points (after formulation, one week, two weeks and six weeks) for the 9V DPs (without aggregated MPL).

FIG. 3: Distribution of the particle sizes of the different 9V DP formulations (with or w/o aggregated MPL) measured by SLS one day after their formulation. The average of 5 measurements per sample after formulation is shown.

FIG. 4: Distribution of the particle sizes of CERVARIX, GARDASIL-9 and the 9V DP (without MPL) measured by SLS. The average of 5 measurements per sample is shown.

FIG. 5: Distribution of the particle sizes of the different 9V DP formulations measured by SLS. The average of 5 measurements per sample is shown.

FIG. 6: Relative potency computed with MPL ref 10 μg/mL depending on MPL concentration for each sample.

FIG. 7: Bioassay measuring the MPL activity of the different 9V DP formulations, measured by quantifying the TNF-α response. Relative potency computed with MPL ref 10 μg/mL depending on MPL concentration for each sample grouped by MPL concentration.

FIG. 8: Bioassay measuring the MPL activity of the different 9V DP formulations, measured by quantifying the TNF-α response. The results have been normalized to the value of the MPL bulk control that has been included in the measurements, resulting in the Relative Potency values.

### LIST OF SEQUENCES

SEQ ID NO: 1—full length HPV 6 L1 amino acid sequence (GENBANK accession number: AAC80442.1)

SEQ ID NO: 2—truncated HPV 6 L1 amino acid sequence

SEQ ID NO: 3—full length HPV 11 L1 amino acid sequence (UNIPROT accession number: P04012.1)

SEQ ID NO: 4—truncated HPV 11 L1 amino acid sequence

SEQ ID NO: 5—full length HPV 16 L1 amino acid sequence (GENBANK accession number: AGC65525.1)

SEQ ID NO: 6—truncated HPV 16 L1 amino acid sequence

SEQ ID NO: 7—full length HPV 18 L1 amino acid sequence (GENBANK accession number: AAQ92369.1)

SEQ ID NO: 8—truncated HPV 18 L1 amino acid sequence

SEQ ID NO: 9—full length HPV 31 L1 amino acid sequence (GENBANK accession number: AAA46956.1)

SEQ ID NO: 10—truncated HPV 31 L1 amino acid sequence

SEQ ID NO: 11—full length HPV 33 L1 amino acid sequence (GENBANK accession number: ACV84009.1)

SEQ ID NO: 12—truncated HPV 33 L1 amino acid sequence

SEQ ID NO: 13—full length HPV 45 L1 amino acid sequence (GENBANK accession number: AAY86494.1)

SEQ ID NO: 14—truncated HPV 45 L1 amino acid sequence

SEQ ID NO: 15—full length HPV 52 L1 amino acid sequence (GENBANK accession number: ACM66882.1)

SEQ ID NO: 16—truncated HPV 52 L1 amino acid sequence

SEQ ID NO: 17—full length HPV 58 L1 amino acid sequence (GENBANK accession number: ACM66884)

SEQ ID NO: 18—truncated HPV 58 L1 amino acid sequence

SEQ ID NO: 19—HPV 6 L1 amino acid sequence

SEQ ID NO: 20—HPV 11 L1 amino acid sequence

SEQ ID NO: 21—HPV 16 L1 amino acid sequence

SEQ ID NO: 22—HPV 18 L1 amino acid sequence

SEQ ID NO: 23—HPV 31 L1 amino acid sequence

SEQ ID NO: 24—HPV 33 L1 amino acid sequence

SEQ ID NO: 25—HPV 45 L1 amino acid sequence

SEQ ID NO: 26—HPV 52 L1 amino acid sequence

SEQ ID NO: 27—HPV 58 L1 amino acid sequence

SEQ ID NO: 28—HPV 16 L1 amino acid sequence

SEQ ID NO: 29—HPV 18 L1 amino acid sequence

SEQ ID NO: 30—full length HPV 26 L1 amino acid sequence (UNIPROT kB accession number: P36735.1)

SEQ ID NO: 31—full length HPV 35 L1 amino acid sequence (GENBANK accession number: QJD38251.1)

SEQ ID NO: 32—full length HPV 39 L1 amino acid sequence (GENBANK accession number: CAD1814503.1)

SEQ ID NO: 33—full length HPV 51 L1 amino acid sequence (GENBANK accession number: ARQ82736.1)

SEQ ID NO: 34—full length HPV 53 L1 amino acid sequence (GENBANK accession number: ACX32368.1)

SEQ ID NO: 35—full length HPV 56 L1 amino acid sequence (GENBANK accession number: CAD1814189.1)

SEQ ID NO: 36—full length HPV 59 L1 amino acid sequence (GENBANK accession number: AGU90696.1)

SEQ ID NO: 37—full length HPV 66 L1 amino acid sequence (GENBANK accession number: ALT54954.1)

SEQ ID NO: 38—full length HPV 67 L1 amino acid sequence (GENBANK accession number: CAD1814012.1)

SEQ ID NO: 39—full length HPV 68 L1 amino acid sequence (GENBANK accession number: ACX32384.1)

SEQ ID NO: 40—full length HPV 70 L1 amino acid sequence (GENBANK accession number: AGU90878.1)

SEQ ID NO: 41—full length HPV 73 L1 amino acid sequence (GENBANK accession number: CAA63887.1)

SEQ ID NO: 42—full length HPV 82 L1 amino acid sequence (GENBANK accession number: BAA90742.1)

### DETAILED DESCRIPTION OF THE INVENTION

The present invention is based on a counterintuitive finding by the inventors that a method for the preparation of an adjuvanted HPV vaccine where HPV antigens pre-adsorbed onto a metallic salt are combined with a TLR4 ligand which has not been pre-adsorbed onto a metallic salt (“free TLR4 ligand” or “non adsorbed TLR4 ligand”), are advantageous. Indeed, the inventors have surprisingly found that this method leads to enhanced TLR4 ligand biological activity as compared to the method disclosed in WO 00/23105 where the TLR4 ligand is pre-adsorbed onto a metallic salt prior to being combined with the HPV antigens. This improved formulation method is referred to herein as “in-line” formulation/production. This “in-line” method goes against the teaching of WO 00/23105 according to which the TLR4 ligand needs to be pre-adsorbed on a metallic salt prior to being combined with the pre-adsorbed HPV antigens, in order to avoid difficulties during quality control (QC) for the assessment of the proper adsorption of the TLR4 ligand and of the antigen.

In one aspect, the present invention provides a method for the preparation of a HPV vaccine composition comprising the steps of (i) adsorbing one or more HPV antigen(s) on a metallic salt and, (ii) adding a non adsorbed glycolipid based TLR4 ligand to the mixture obtained in (i).

As used herein, an “in-line” process or method is a process or method where antigens, in particular HPV antigens, are combined with a TLR4 ligand which has not been pre-adsorbed onto a metallic salt. The antigens may be pre-adsorbed onto a metallic salt.

As used herein, a “salt” is a chemical compound consisting of an ionic assembly of cations and anions, and a “metallic salt” is a salt comprising a metallic ion. Preferred metallic salt are aluminum salts comprising A³⁺.

As used herein, a “non adsorbed” (or “free” or “not pre-adsorbed”) glycolipid based TLR4 ligand is a glycolipid based TLR4 ligand which has not been pre-adsorbed on a metallic salt.

As used herein, a “TLR4 ligand” is a molecule capable of binding to TLR4 (Toll Like Receptor 4). TLR4 is a transmembrane protein, member of the toll-like receptor family, which belongs to the pattern recognition receptor (PRR) family. It is most well-known for recognizing lipopolysaccharide (LPS), a component present in many Gram-negative bacteria and some Gram-positive bacteria. Upon activation, TLR4 triggers the production of mature IL18. IL18 then drives the production of INF-γ by innate cells including natural killer cells (NK) and neutrophils, as well as memory CD8 T cells, that in turn promote TH1 immunity. Nontoxic TLR4 ligands can thus be used as adjuvants.

As used herein, a “glycolipid based” TLR4 ligand is a non toxic TLR4 ligand based on an oligosaccharide structure covalently linked to one or more lipid chains. Such a TLR4 ligand is suitable for use as an adjuvant in a vaccine composition. Glycolipid based TLR4 ligand can be derived from bacterial LPS, Lipid-A or chemically synthesized. Suitable glycolipid based TLR4 ligands include
disaccharide glycolipids, such as MPL (or 3D-MPL), GLA, SLA and OM-174,
monosaccharide glycolipids such as CCL-34, RC-529, PET Lipid A and ONO-4007, CRX601, and
lipooligosaccharides such as dLOS,
   the structures of which are shown below:

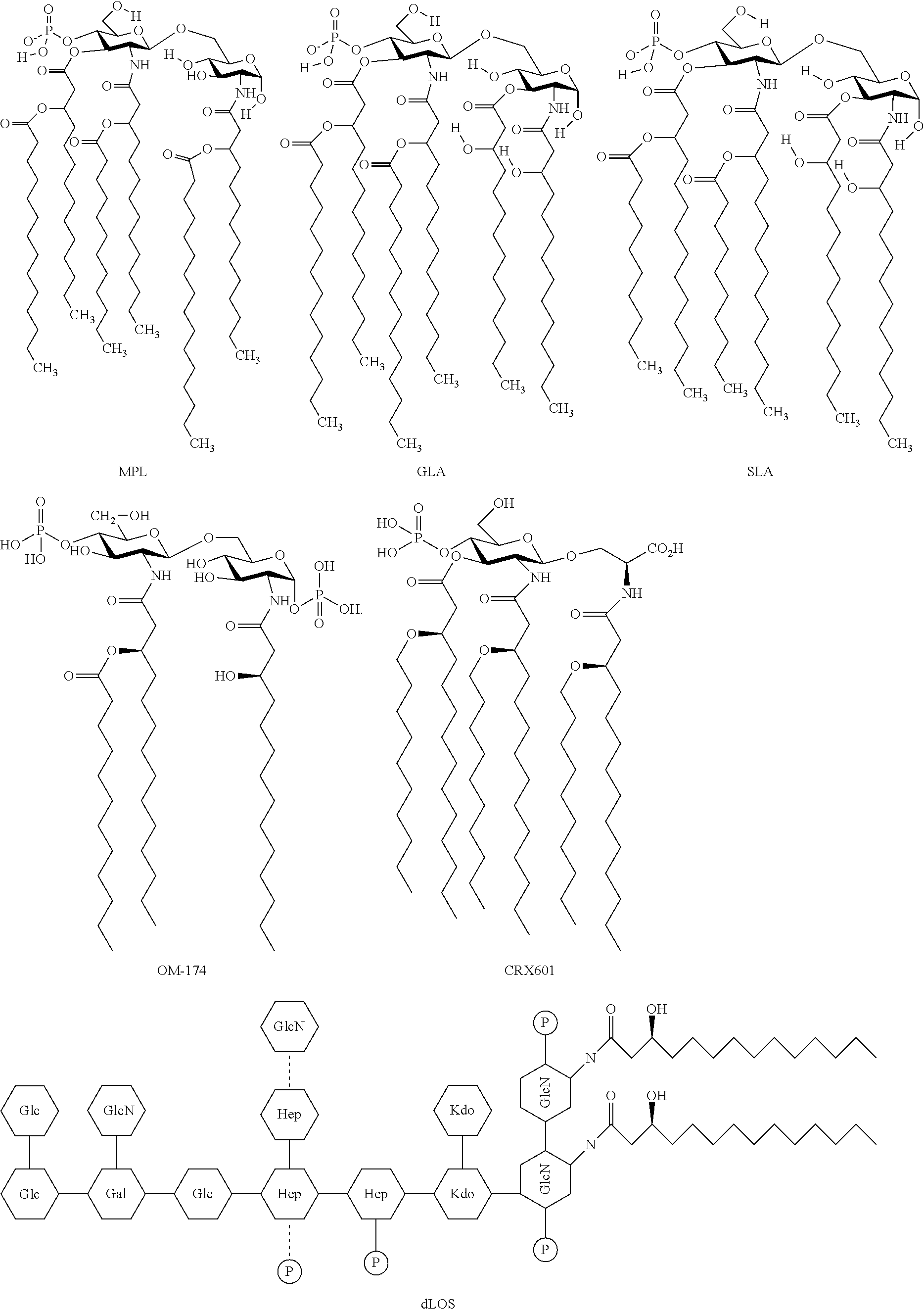

MPL (3-Deacylated monophoshoryl lipid A) is commercialized by GlaxoSmithKline Biologicals, and is referred to herein as MPL or 3D-MPL. See, for example, U.S. Pat. Nos. 4,436,727; 4,877,611; 4,866,034 and 4,912,094. 3D-MPL primarily promotes CD4+ T cell responses with an IFN-γ (Th1) phenotype. 3D-MPL can be produced according to the methods disclosed in GB2220211 A.

Chemically, it is a mixture of 3-deacylated monophosphoryl lipid A with 3, 4, 5 or 6 acylated chains. In the compositions of the present invention small particle 3D-MPL can be used. Small particle 3D-MPL has a particle size such that it can be sterile-filtered through a 0.22 μm filter. Such preparations are described in WO94/21292.

GLA (glucopyranosyl lipid adjuvant) and SLA (second-generation lipid adjuvant) are two synthetic analogs of detoxified MPL. The structures of GLA and SLA are based on molecules in MPL except that in GLA the hexa-acyl chain is at the 3 position, whereas MPL is 3-deacylated. SLA is a version of GLA where the length of some acyl chains is different. (Reed et al., 2016⁵).

OM-174 is a purified water soluble dephosphorylated and triacetylated lipid A derived from E. coli. This compound is currently being developed as an adjuvant for therapeutic vaccination, and mainly for cancer applications (D'Agostini et al. 2005⁹).

CCL-34 is a synthetic glycolipid that induces the activation of macrophages and the maturation of dendritic cells in a TLR4-dependent manner (Chou et al., 2020⁷).

RC-529 is a chemically obtained MPL mimic, which combined with aluminum oxyhydroxide formed the adjuvant component of a hepatitis B virus vaccine licensed in Argentina (Fox et al. 2017⁴, Shimoyama and Fukase, 2020⁸);

PET Lipid A is a synthetic hexa-acylated monosaccharide monophosphorylated TLR4 ligand. The combination of a liposomal formulation of this compound and a cancer antigen was tolerated in phase I clinical trial (Fox et al. 2017⁴, Hamdy et al. 2007¹⁰, Kawther et al.¹³).

ONO-4007 is a synthetic lipid A derivative featuring a monosaccharide triacyl structure, with two of the chains terminating in benzene rings and the head group phosphate replaced by a sulfate. A phase I immunotherapy trial was reported with this compound in 2002 (Kuramitsu et al. 1997¹¹). CRX601 is an alkyl glucosaminide phosphate (AGP) described in WO98/50399 and U.S. Pat. No. 6,303,347 (processes for preparation of AGPs are also described).

dLOS (deacylated lipooligosaccharide, also referred to as “CIA05”) consists of a core oligosaccharide lacking the terminal glucose residue, a glucosamine disaccharide with two phosphate groups, and two N-linked acyl groups. dLOS induces Th1, Th2, and Th17-type immune responses in a dose dependent manner (Eun et al. 2014⁶, Seo Ri Wui et al.¹⁴).

In one embodiment, the glycolipid based TLR4 ligand is selected from disaccharide glycolipids, monosaccharide glycolipids and lipooligosaccharides. In a preferred embodiment, the glycolipid-based TLR4 ligand is selected from MPL, GLA, SLA, OM-174, CCL-34, RC-529, PET-Lipid A, ONO-4007, CRX601 and dLOS. In a preferred embodiment, the glycolipid based TLR4 ligand is MPL.

In one embodiment, the one or more HPV antigen(s) are selected from HPV late proteins L1 and L2, chimeric L1 proteins, chimeric L1/L2 proteins, and immunogenic fragments thereof. A “late protein” (L) is a viral protein that is formed after the virus replication. It usually surrounds the genetic material of a virus as a capsid. L1 proteins are the major proteins of the HPV capsid, and L2 proteins are the minor proteins of the HPV capsid. A chimeric L1 protein is a protein comprising parts of an L1 protein from a first HPV type and parts of an L1 protein from a second HPV type wherein the first and second HPV types are different. A chimeric L1/L2 protein is a protein comprising parts of an L1 HPV protein and parts of an L2 HPV protein, for example an L1 HPV protein expressing one or more conserved L2 epitopes.

Exemplary HPV L1 sequences are shown in table 1.

TABLE 1
Exemplary HPV L1 sequences

| Protein | SEQ ID NO | Sequence |
|---|---|---|
| HPV6 L1 | 1 | MWRPSDSTVYVPPPNPVSKVVATDAYVTRTNIFYHASSSRLLAVGHPYF |
| | | SIKRANKTVVPKVSGYQYRVFKVVLPDPNKFALPDSSLEDPTTQRLVWA |
| | | CTGLEVGRGQPLGVGVSGHPFLNKYDDVENSGSGGNPGQDNRVNVGMDY |
| | | KQTQLCMVGCAPPLGEHWGKGKQCTNTPVQAGDCPPLELITSVIQDGDM |
| | | VDTGFGAMNFADLQTNKSDVPIDICGTTCKYPDYLQMAADPYGDRLFFF |
| | | LRKEQMFARHFFNRAGEVGEPVPDTLIIKGSGNRTSVGSSIYVNTPSGS |
| | | LVSSEAQLENKPYWLQKAQGHNNGICWGNQLFVTVVDTTRSTNMTLCAS |
| | | VTTSSTYTNSDYKEYMRHVEEYDLQFIFQLCSITLSAEVMAYIHTMNPS |
| | | VLEDWNFGLSPPPNGTLEDTYRYVQSQAITCQKPTPEKQKPDPYKNLSE |
| | | WEVNLKEKESSELDQYPLGRKELLQSGYRGRSSIRTGVKRPAVSKASAA |
| | | PKRKRAKTKR |
| | 2 | MDSTVYVPPPNPVSKVVATDAYVTRTNIFYHASSSRLLAVGHPYESIKR |
| | | ANKTVVPKVSGYQYRVFKVVLPDPNKFALPDSSLEDPTTQRLVWACTGL |
| | | EVGRGQPLGVGVSGHPFLNKYDDVENSGSGGNPGQDNRVNVGMDYKQTQ |
| | | LCMAGCAPPLGEHWGKGKQCTNTPVQAGDCPPLELITSVIQDGDMVDTG |
| | | FGAMNFADLQTNKSDVPIDICGTTCKYPDYLQMAADPYGDRLFFELRKE |
| | | QMFARHFFNRAGEVGEPVPDTLIIKGSGNRTSVGSSIYVNTPSGSLVSS |
| | | EAQLFNKPYWLQKAQGHNNGICWGNQLFVTVVDTTRSTNMTLCASVTTS |
| | | STYTNSDYKEYMRHVEEYDLQFIFQLCSITLSAEVVAYIHTMNPSVLED |
| | | WNFGLSPPPNGTLEDTYRYVQSQAITCQKPTPEKQKPDPYKNLSFWEVN |
| | | LKEKESSELDQYPLGRKELLQSGYRGRSSIRTGVKRPAVSKASAAPKRK |
| | | RAKTKR |
| | 19 | MWRPSDSTVYVPPPNPVSKVVATDAYVTRTNIFYHASSSRLLAVGHPYF |
| | | SIKRANKTVVPKVSGYQYRVFKVVLPDPNKFALPDSSLEDPTTQRLVWA |
| | | CTGLEVGRGQPLGVGVSGHPFLNKYDDVENSGSGGNPGQDNRVNVGMDY |
| | | KQTQLCMAGCAPPLGEHWGKGKQCTNTPVQAGDCPPLELITSVIQDGDM |
| | | VDTGFGAMNFADLQTNRSDVPIDICGTTCKYPDYLQMAADPYGDRLFFF |
| | | LRKEQMFARHFFNRAGEVGEPVPDTLIIKGSGNRTSVGSSIYVNTPSGS |
| | | LVSSEAQLENKPYWLQKAQGHNNGICWGNQLFVTVVDTTRSTNMTLCAS |
| | | VTTSSTYTNSDYKEYMRHVEEYDLQFIFQLCSITLSAEVMAYIHTMNPS |
| | | VLEDWNFGLSPPPNGTLEDTYRYVQSQAITCQKPTPEKQKPDPYKNLSF |
| | | WEVNLKEKESSELDQYPLGRKELLQSGYRGRSSIRTGVKRPAVSKASAA |
| | | PKRKRAKTKR |
| HPV11 | 3 | MWRPSDSTVYVPPPNPVSKVVATDAYVKRINIFYHASSSRLLAVGHPYY |
| L1 | | SIKKVNKTVVPKVSGYQYRVFKVVLPDPNKFALPDSSLEDPTTQRLVWA |
| | | CTGLEVGRGQPLGVGVSGHPLLNKYDDVENSGGYGGNPGQDNRVNVGMD |
| | | YKQTQLCMVGCAPPLGEHWGKGTQCSNTSVQNGDCPPLELITSVIQDGD |
| | | MVDTGFGAMNFADLQTNKSDVPLDICGTVCKYPDYLQMAADPYGDRLFF |
| | | YLRKEQMFARHFFNRAGTVGEPVPDDLLVKGGNNRSSVASSIYVHTPSG |
| | | SLVSSEAQLENKPYWLQKAQGHNNGICWGNHLFVTVVDTTRSTNMTLCA |
| | | SVSKSATYTNSDYKEYMRHVEEFDLQFIFQLCSITLSAEVMAYIHTMNP |
| | | SVLEDWNFGLSPPPNGTLEDTYRYVQSQAITCQKPTPEKEKQDPYKDMS |
| | | FWEVNLKEKESSELDQFPLGRKELLQSGYRGRTSARTGIKRPAVSKPST |
| | | APKRKRTKTKK |
| | 4 | MSDSTVYVPPPNPVSKVVATDAYVKRTNIFYHASSSRLLAVGHPYYSIK |
| | | KVNKTVVPKVSGYQYRVFKVVLPDPNKFALPDSSLEDPTTQRLVWACTG |
| | | LEVGRGQPLGVGVSGHPLLNKYDDVENSGGYGGNPGQDNRVNVGMDYKQ |
| | | TQLCMVGCAPPLGEHWGKGTQCSNTSVQNGDCPPLELITSVIQDGDMVD |
| | | TGFGAMNFADLQTNKSDVPLDICGTVCKYPDYLQMAADPYGDRLFFYLR |
| | | KEQMFARHFFNRAGTVGEPVPDDLLVKGGNNRSSVASSIYVHTPSGSLV |
| | | SSEAQLENKPYWLQKAQGHNNGICWGNHLFVTVVDTTRSTNMTLCASVS |
| | | KSATYTNSDYKEYMRHVEEFDLQFIFQLCSITLSAEVMAYIHTMNPSVL |
| | | EDWNFGLSPPPNGTLEDTYRYVQSQAITCQKPTPEKEKQDPYKDMSEWE |
| | | VNLKEKESSELDQFPLGRKELLQSGYRGRTSARTGIKRPAVSKPSTAPK |
| | | RKRTKTKK |
| | 20 | MWRPMSDSTVYVPPPNPVSKVVATDAYVKRTNIFYHASSSRLLAVGHPY |
| | | YSIKKVNKTVVPKVSGYQYRVFKVVLPDPNKFALPDSSLEDPTTQRLVW |
| | | ACTGLEVGRGQPLGVGVSGHPLLNKYDDVENSGGYGGNPGQDNRVNVGM |
| | | DYKQTQLCMVGCAPPLGEHWGKGTQCSNTSVQNGDCPPLELITSVIQDG |
| | | DMVDTGFGAMNFADLQTNKSDVPLDICGTVCKYPDYLQMAADPYGDRLF |
| | | FYLRKEQMFARHEENRAGTVGEPVPDDLLVKGGNNRSSVASSIYVHTPS |
| | | GSLVSSEAQLENKPYWLQKAQGHNNGICWGNHLFVTVVDTTRSTNMTLC |
| | | ASVSKSATYTNSDYKEYMRHVEEFDLQFIFQLCSITLSAEVMAYIHTMN |
| | | PSVLEDWNFGLSPPPNGTLEDTYRYVQSQAITCQKPTPEKEKQDPYKDM |
| | | SFWEVNLKEKESSELDQFPLGRKELLQSGYRGRTSARTGIKRPAVSKPS |
| | | TAPKRKRTKTKK |
| HPV16 | 5 | MEVTFIYILVITCYENDVNVYHIFFQMSLWLPSEATVYLPPVPVSKVVS |
| L1 | | TDEYVARTNIYYHAGTSRLLAVGHPYFPIKKPNNNKILVPKVSGLQYRV |
| | | FRIHLPDPNKFGFPDTSFYNPDTQRLVWACVGVEVGRGQPLGVGISGHP |
| | | LLNKLDDTENASAYAANAGVDNRECISMDYKQTQLCLIGCKPPIGEHWG |
| | | KGSPCTNVAVNPGDCPPLELINTVIQDGDMVDTGFGAMDFTTLQANKSE |
| | | VPLDICTSICKYPDYIKMVSEPYGDSLFFYLRREQMEVRHLENRAGAVG |
| | | ENVPDDLYIKGSGSTANLASSNYFPTPSGSMVTSDAQIENKPYWLQRAQ |
| | | GHNNGICWGNQLFVTVVDTTRSTNMSLCAAISTSETTYKNTNFKEYLRH |
| | | GEEYDLQFIFQLCKITLTADVMTYIHSMNSTILEDWNFGLQPPPGGTLE |
| | | DTYRFVTSQAIACQKHTPPAPKEDPLKKYTFWEVNLKEKESADLDQFPL |
| | | GRKELLQAGLKAKPKFTLGKRKATPTTSSTSTTAKRKKRKL |
| | 6 | MLPSEATVYLPPVPVSKVVSTDEYVARTNIYYHAGTSRLLAVGHPYFPI |
| | | KKPNNNKILVPKVSGLQYRVFRIHLPDPNKFGFPDTSFYNPDTQRLVWA |
| | | CVGVEVGRGQPLGVGISGHPLLNKLDDTENASAYAANAGVDNRECISMD |
| | | YKQTQLCLIGCKPPIGEHWGKGSPCTNVAVNPGDCPPLELINTVIQDGD |
| | | MVDTGFGAMDFTTLQANKSEVPLDICTSICKYPDYIKMVSEPYGDSLFF |
| | | YLRREQMFVRHLFNRAGAVGDNVPDDLYIKGSGSTANLASSNYFPTPSG |
| | | SMVTSDAQIFNKPYWLQRAQGHNNGICWGNQLFVTVVDTTRSTNMSLCA |
| | | AISTSETTYKNTNFKEYLRHGEEYDLQFIFQLCKITLTADIMTYIHSMN |
| | | STILEDWNFGLQPPPGGTLEDTYREVTSQAIACQKHTPPAPKEDPLKKY |
| | | TFWEVNLKEKFSADLDQFPLGRKELLQAGLEAKPKFTLGKRKATPTTSS |
| | | TSTTAKRKKRKL |
| | 21 | MSLWLPSEATVYLPPVPVSKVVSTDEYVARTNIYYHAGTSRLLAVGHPY |
| | | FPIKKPNNNKILVPKVSGLQYRVFRIHLPDPNKFGFPDTSFYNPDTQRL |
| | | VWACVGVEVGRGQPLGVGISGHPLLNKLDDTENASAYAANAGVDNRECI |
| | | SMDYKQTQLCLIGCKPPIGEHWGKGSPCTNVAVNPGDCPPLELINTVIQ |
| | | DGDMVDTGFGAMDFTTLQANKSEVPLDICTSICKYPDYIKMVSEPYGDS |
| | | LFFYLRREQMFVRHLENRAGAVGENVPDDLYIKGSGSTANLASSNYFPT |
| | | PSGSMVTSDAQIFNKPYWLQRAQGHNNGICWGNQLFVTVVDTTRSTNMS |
| | | LCAAISTSETTYKNTNFKEYLRHGEEYDLQFIFQLCKITLTADVMTYIH |
| | | SMNSTILEDWNFGLQPPPGGTLEDTYRFVTSQAIACQKHTPPAPKEDPL |
| | | KKYTFWEVNLKEKFSADLDQFPLGRKELLQAGLKAKPKFTLGKRKATPT |
| | | TSSTSTTAKRKKRKL |
| | 28 | MSLWLPSEATVYLPPVPVSKVVSTDEYVARTNIYYHAGTSRLLAVGHPY |
| | | FPIKKPNNNKILVPKVSGLQYRVFRIHLPDPNKFGFPDTSFYNPDTQRL |
| | | VWACVGVEVGRGQPLGVGISGHPLLNKLDDTENASAYAANAGVDNRECI |
| | | SMDYKQTQLCLIGCKPPIGEHWGKGSPCTNVAVNPGDCPPLELINTVIQ |
| | | DGDMVDTGFGAMDFTTLQANKSEVPLDICTSICKYPDYIKMVSEPYGDS |
| | | LFFYLRREQMFVRHLENRAGAVGENVPDDLYIKGSGSTANLASSNYFPT |
| | | PSGSMVTSDAQIFNKPYWLQRAQGHNNGICWGNQLFVTVVDTTRSTNMS |
| | | LCAAISTSETTYKNTNFKEYLRHGEEYDLQFIFQLCKITLTADVMTYIH |
| | | SMNSTILEDWNFGLQPPPGGTLEDTYRFVTSQAIACQKHTPPAPKEDPL |
| | | KKYTFWEVNLKEKFSADLDQFPLGRKELLQ |
| HPV18 | 7 | MALWRPSDNTVYLPPPSVARVVNTDDYVTRTSIFYHAGSSRLLTVGNPY |
| L1 | | FRVPAGGGNKQDIPKVSAYQYRVERVQLPDPNKFGLPDTSIYNPETQRL |
| | | VWACAGVEIGRGQPLGVGLSGHPFYNKLDDTESSHAATSNVSEDVRDNV |
| | | SVDYKQTQLCILGCAPAIGEHWAKGTACKSRPLSQGDCPPLELKNTVLE |
| | | DGDMVDTGYGAMDFSTLQDTKCEVPLDICQSICKYPDYLQMSADPYGDS |
| | | MFFCLRREQLFARHEWNRAGTMGDTVPQSLYIKGTGMRASPGSCVYSPS |
| | | PSGSIVTSDSQLENKPYWLHKAQGHNNGVCWHNQLFVTVVDTTRSTNLT |
| | | ICASTQSPVPGQYDATKFKQYSRHVEEYDLQFIFQLCTITLTADVMSYI |
| | | HSMNSSILEDWNFGVPPPPTTSLVDTYREVQSVAITCQKDAAPAENKDP |
| | | YDKLKFWNV |
| | 8 | MRPSDNTVYLPPPSVARVVNTDDYVTRTSIFYHAGSSRLLTVGNPYFRV |
| | | PAGGGNKQDIPKVSAYQYRVFRVQLPDPNKFGLPDTSIYNPETQRLVWA |
| | | CAGVEIGRGQPLGVGLSGHPFYNKLDDTESSHAATSNVSEDVRDNVSVD |
| | | YKQTQLCILGCAPAIGEHWAKGTACKSRPLSQGDCPPLELKNTVLEDGD |
| | | MVDTGYGAMDFSTLQDTKCEVPLDICQSICKYPDYLQMSADPYGDSMFF |
| | | CLRREQLFARHEWNRAGTMGDTVPQSLYIKGTGMRASPGSCVYSPSPSG |
| | | SIVTSDSQLENKPYWLHKAQGHNNGVCWHNQLFVTVVDTTRSTNLTICA |
| | | STQSPVPGQYDATKFKQYSRHVEEYDLQFIFQLCTITLTADVMSYIHSM |
| | | NSSILEDWNFGVPPPPTTSLVDTYREVQSVAIACQKDAAPAENKDPYDK |
| | | LKFWNVDLKEKFSLDLDQYPLGRKFLVQAGLRRKPTIGPRKRSAPSATT |
| | | ASKPAKRVRVRARK |
| | 22 | MALWRPSDNTVYLPPPSVARVVNTDDYVTRTSIFYHAGSSRLLTVGNPY |
| | | FRVPAGGGNKQDIPKVSAYQYRVERVQLPDPNKEGLPDNSIYNPETQRL |
| | | VWACAGVEIGRGQPLGVGLSGHPFYNKLDDTESSHAATSNVSEDVRDNV |
| | | SVDYKQTQLCILGCAPAIGEHWAKGTACKSRPLSQGDCPPLELKNTVLE |
| | | DGDMVDTGYGAMDFSTLQDTKCEVPLDICQSICKYPDYLQMSADPYGDS |
| | | MFFCLRREQLFARHEWNRAGTMGDTVPQSLYIKGTGMRASPGSCVYSPS |
| | | PSGSIVTSDSQLENKPYWLHKAQGHNNGICWHNQLFVTVVDTTRSTNLT |
| | | ICASTQSPVPGQYDATKFKQYSRHVEEYDLQFIFQLCTITLTADVMSYI |
| | | HSMNSSILEDWNFGVPPPPTTSLVDTYREVQSVAITCQKDAAPAENKDP |
| | | YDKLKFWNVDLKEKESLDLDQYPLGRKELVQAGLRRKPTIGPRKRSAPS |
| | | ATTSSKPAKRVRVRARK |
| | 29 | MALWRPSDNTVYLPPPSVARVVNTDDYVTRTSIFYHAGSSRLLTVGNPY |
| | | FRVPAGGGNKQDIPKVSAYQYRVERVQLPDPNKFGLPDNSIYNPETQRL |
| | | VWACVGVEIGRGQPLGVGLSGHPFYNKLDDTESSHAATSNVSEDVRDNV |
| | | SVDYKQTQLCILGCAPAIGEHWAKGTACKSRPLSQGDCPPLELKNTVLE |
| | | DGDMVDTGYGAMDFSTLQDTKCEVPLDICQSICKYPDYLQMSADPYGDS |
| | | MFFCLRREQLFARHEWNRAGTMGDTVPPSLYIKGTGMRASPGSCVYSPS |
| | | PSGSIVTSDSQLENKPYWLHKAQGHNNGVCWHNQLFVTVVDTTRSTNLT |
| | | ICASTQSPVPGQYDATKFKQYSRHVEEYDLQFIFQLCTITLTADVMSYI |
| | | HSMNSSILEDWNFGVPPPPTTSLVDTYREVQSVAITCQKDAAPAENKDP |
| | | YDKLKFWNVDLKEKFSLDLDQYPLGRKELVQ |
| HPV31 | 9 | MSLWRPSEATVYLPPVPVSKVVSTDEYVTRINIYYHAGSARLLTVGHPY |
| L1 | | YSIPKSDNPKKIVVPKVSGLQYRVFRVRLPDPNKFGFPDTSFYNPETQR |
| | | LVWACVGLEVGRGQPLGVGISGHPLLNKEDDTENSNRYAGGPGTDNREC |
| | | ISMDYKQTQLCLLGCKPPIGEHWGKGSPCSNNAITPGDCPPLELKNSVI |
| | | QDGDMVDTGFGAMDFTALQDTKSNVPLDICNSICKYPDYLKMVAEPYGD |
| | | TLFFYLRREQMFVRHFFNRSGTVGESVPTDLYIKGSGSTATLANSTYFP |
| | | TPSGSMVTSDAQIFNKPYWMQRAQGHNNGICWGNQLFVTVVDTTRSTNM |
| | | SVCAAIANSDTTFKSSNFKEYLRHGEEFDLQFIFQLCKITLSADIMTYI |
| | | HSMNPAILEDWNFGLTTPPSGSLEDTYRFVTSQAITCQKTAPQKPKEDP |
| | | FKDYVFWEVNLKEKFSADLDQFPLGRKELLQAGYRARPKFKAGKRSAPS |
| | | ASTTTPAKRKKTKK |
| | 10 | MSLWRPSEATVYLPPVPVSKVVSTDEYVTRINIYYHAGSARLLTVGHPY |
| | | YSIPKSDNPKKIVVPKVSGLQYRVFRVRLPDPNKFGFPDTSFYNPETQR |
| | | LVWACVGLEVGRGQPLGVGISGHPLLNKEDDTENSNRYAGGPGTDNREC |
| | | ISMDYKQTQLCLLGCKPPIGEHWGKGSPCSNNAITPGDCPPLELKNSVI |
| | | QDGDMVDTGFGAMDFTALQDTKSNVPLDICNSICKYPDYLKMVAEPYGD |
| | | TLFFYLRREQMFVRHFFNRSGTVGESVPTDLYIKGSGSTATLANSTYFP |
| | | TPSGSMVTSDAQIFNKPYWMQRAQGHNNGICWGNQLFVTVVDTTRSTNM |
| | | SVCAAIANSDTTFKSSNFKEYLRHGEEFDLQFIFQLCKITLSADIMTYI |
| | | HSMNPAILEDWNFGLTTPPSGSLEDTYRFVTSQAITCQKTAPQKPKEDP |
| | | FKDYVFWEVNLKEKESADLDQFPLGRKELLQAGYRARPKFKAGKRSAPS |
| | | ASTTTPAKRKKTKK |
| | 23 | MSLWRPSEATVYLPPVPVSKVVSTDEYVTRTNIYYHAGSARLLTVGHPY |
| | | YSIPKSDNPKKIVVPKVSGLQYRVFRVRLPDPNKFGFPDTSFYNPETQR |
| | | LVWACVGLEVGRGQPLGVGISGHPLLNKEDDTENSNRYAGGPGTDNREC |
| | | ISMDYKQTQLCLLGCKPPIGEHWGKGSPCSNNAITPGDCPPLELKNSVI |
| | | QDGDMVDTGFGAMDFTALQDTKSNVPLDICNSICKYPDYLKMVAEPYGD |
| | | TLFFYLRREQMFVRHFFNRSGTVGESVPTDLYIKGSGSTATLANSTYFP |
| | | TPSGSMVTSDAQIENKPYWMQRAQGHNNGICWGNQLFVTVVDTTRSTNM |
| | | SVCAAIANSDTTFKSSNFKEYLRHGEEFDLQFIFQLCKITLSADIMTYI |
| | | HSMNPAILEDWNFGLTTPPSGSLEDTYREVTSQAITCQKSAPQKPKEDP |
| | | FKDYVFWEVNLKEKFSADLDQFPLGRKELLQAGYRARPKFKAGKRSAPS |
| | | ASTTTPAKRKKTKK |
| HPV33 | 11 | MSVWRPSEATVYLPPVPVSKVVSTDEYVSRTSIYYYAGSSRLLAVGHPY |
| L1 | | FSIKNPTNAKKLLVPKVSGLQYRVFRVRLPDPNKFGFPDTSFYNPDTQR |
| | | LVWACVGLEIGRGQPLGVGISGHPLLNKEDDTETGNKYPGQPGADNREC |
| | | LSMDYKQTQLCLLGCKPPTGEHWGKGVACTNAAPANDCPPLELINTIIE |
| | | DGDMVDTGFGCMDFKTLQANKSDVPIDICGSTCKYPDYLKMTSEPYGDS |
| | | LFFFLRREQMFVRHFFNRAGTLGEAVPDDLYIKGSGTTASIQSSAFFPT |
| | | PSGSMVTSESQLENKPYWLQRAQGHNNGICWGNQVFVTVVDTTRSTNMT |
| | | LCTQVTSDSTYKNENFKEYIRHVEEYDLQFVFQLCKVTLTAEVMTYIHA |
| | | MNPDILEDWQFGLTPPPSASLQDTYRFVTSQAITCQKTVPPKEKEDPLG |
| | | KYTFWEVDLKEKFSADLDQFPLGRKELLQAGLKAKPKLKRAAPTSTRTS |
| | | SAKRKKVKK |
| | 12 | MTVYLPPVPVSKVVSTDEYVSRTSIYYYAGSSRLLAVGHPYESIKNPTN |
| | | AKKLLVPKVSGLQYRVERVRLPDPNKFGFPDTSFYNPDTQRLVWACVGL |
| | | EIGRGQPLGVGISGHPLLNKEDDTETGNKYPGQPGADNRECLSMDYKQT |
| | | QLCLLGCKPPTGEHWGKGVACTNAAPANDCPPLELINTIIEDGDMVDTG |
| | | FGCMDFKTLQANKSDVPIDICGSTCKYPDYLKMTSEPYGDSLFFELRRE |
| | | QMFVRHFFNRAGTLGEAVPDDLYIKGSGTTASIQSSAFFPTPSGSMVTS |
| | | ESQLENKPYWLQRAQGHNNGICWGNQVFVTVVDTTRSTNMTLCTQVTSD |
| | | STYKNENFKEYIRHVEEYDLQFVFQLCKVTLTAEVMTYIHAMNPDILED |
| | | WQFGLTPPPSASLQDTYRFVTSQAITCQKTVPPKEKEDPLGKYTFWEVD |
| | | LKEKFSADLDQFPLGRKELLQAGLKAKPKLKRAAPTSTRTSSAKRKKVK |
| | | K |
| | 24 | MSVWRPSEATVYLPPVPVSKVVSTDEYVSRTSIYYYAGSSRLLAVGHPY |
| | | FSIKNPTNAKKLLVPKVSGLQYRVFRVRLPDPNKFGFPDTSFYNPDTQR |
| | | LVWACVGLEIGRGQPLGVGISGHPLLNKEDDTETGNKYPGQPGADNREC |
| | | LSMDYKQTQLCLLGCKPPTGEHWGKGVACTNAAPANDCPPLELINTIIE |
| | | DGDMVDTGFGCMDFKTLQANKSDVPIDICGSTCKYPDYLKMTSEPYGDS |
| | | LFFELRREQMEVRHFFNRAGTLGEAVPDDLYIKGSGTTASIQSSAFFPT |
| | | PSGSMVTSESQLENKPYWLQRAQGHNNGICWGNQVFVTVVDTTRSTNMT |
| | | LCTQVTSDSTYKNENFKEYIRHVEEYDLQFVFQLCKVTLTAEVMTYIHA |
| | | MNPDILEDWQFGLTPPPSASLQDTYRFVTSQAITCQKTVPPKEKEDPLG |
| | | KYTFWEVDLKEKFSADLDQFPLGRKELLQAGLKAKPKLKRAAPTSTRTS |
| | | SAKRKKVKK |
| HPV45 | 13 | MALWRPSDSTVYLPPPSVARVVSTDDYVSRTSIFYHAGSSRLLTVGNPY |
| L1 | | FRVVPNGAGNKQAVPKVSAYQYRVERVALPDPNKFGLPDSTIYNPETQR |
| | | LVWACVGMEIGRGQPLGIGLSGHPFYNKLDDTESAHAATAVITQDVRDN |
| | | VSVDYKQTQLCILGCVPAIGEHWAKGTLCKPAQLQPGDCPPLELKNTII |
| | | EDGDMVDTGYGAMDFSTLQDTKCEVPLDICQSICKYPDYLQMSADPYGD |
| | | SMFFCLRREQLFARHEWNRAGVMGDTVPTDLYIKGTSANMRETPGSCVY |
| | | SPSPSGSIITSDSQLENKPYWLHKAQGHNNGICWHNQLFVTVVDTTRST |
| | | NLTLCASTQNPVPSTYDPTKFKQYSRHVEEYDLQFIFQLCTITLTAEVM |
| | | SYIHSMNSSILENWNFGVPPPPTTSLVDTYREVQSVAVTCQKDTTPPEK |
| | | QDPYDKLKFWTVDLKEKFSSDLDQYPLGRKELVQAGLRRRPTIGPRKRP |
| | | AASTSTASTASRPAKRVRIRSKK |
| | 14 | MALWRPSDSTVYLPPPSVARVVSTDDYVSRTSIFYHAGSSRLLTVGNPY |
| | | FRVVPNGAGNKQAVPKVSAYQYRVERVALPDPNKFGLPDSTIYNPETQR |
| | | LVWACVGMEIGRGQPLGIGLSGHPFYNKLDDTESAHAATAVITQDVRDN |
| | | VSVDYKQTQLCILGCVPAIGEHWAKGTLCKPAQLQPGDCPPLELKNTII |
| | | EDGDMVDTGYGAMDFSTLQDTKCEVPLDICQSICKYPDYLQMSADPYGD |
| | | SMFFCLRREQLFARHEWNRAGVMGDTVPTDLYIKGTSANMRETPGSCVY |
| | | SPSPSGSIITSDSQLENKPYWLHKAQGHNNGICWHNQLFVTVVDTTRST |
| | | NLTLCASTQNPVPSTYDPTKFKQYSRHVEEYDLQFIFQLCTITLTAEVM |
| | | SYIHSMNSSILENWNFGVPPPPTTSLVDTYRFVQSVAVTCQKDTTPPEK |
| | | QDPYDKLKFWTVDLKEKFSSDLDQYPLGRKELVQAGLRRRPTIGPRKRP |
| | | AASTSTASTASRPAKRVRIRSKK |
| | 25 | MALWRPSDSTVYLPPPSVARVVNTDDYVSRTSIFYHAGSSRLLTVGNPY |
| | | FRVVPSGAGNKQAVPKVSAYQYRVERVALPDPNKFGLPDSTIYNPETQR |
| | | LVWACVGMEIGRGQPLGIGLSGHPFYNKLDDTESAHAATAVITQDVRDN |
| | | VSVDYKQTQLCILGCVPAIGEHWAKGTLCKPAQLQPGDCPPLELKNTII |
| | | EDGDMVDTGYGAMDFSTLQDTKCEVPLDICQSICKYPDYLQMSADPYGD |
| | | SMFFCLRREQLFARHEWNRAGVMGDTVPTDLYIKGTSANMRETPGSCVY |
| | | SPSPSGSITTSDSQLENKPYWLHKAQGHNNGICWHNQLFVTVVDTTRST |
| | | NLTLCASTQNPVPNTYDPTKFKHYSRHVEEYDLQFIFQLCTITLTAEVM |
| | | SYIHSMNSSILENWNFGVPPPPTTSLVDTYRFVQSVAVTCQKDTTPPEK |
| | | QDPYDKLKFWTVDLKEKFSSDLDQYPLGRKELVQAGLRRRPTIGPRKRP |
| | | AASTSTASRPAKRVRIRSKK |
| HPV52 | 15 | MVQILFYILVIFYYVAGVNVFHIFLQMSVWRPSEATVYLPPVPVSKVVS |
| L1 | | TDEYVSRTSIYYYAGSSRLLTVGHPYFSIKNTSSGNGKKVLVPKVSGLQ |
| | | YRVFRIKLPDPNKFGFPDTSFYNPETQRLVWACTGLEIGRGQPLGVGIS |
| | | GHPLLNKEDDTETSNKYAGKPGIDNRECLSMDYKQTQLCILGCKPPIGE |
| | | HWGKGTPCNNNSGNPGDCPPLQLINSVIQDGDMVDTGFGCMDENTLQAS |
| | | KSDVPIDICSSVCKYPDYLQMASEPYGDSLFFFLRREQMFVRHFENRAG |
| | | TLGDPVPGDLYIQGSNSGNTATVQSSAFFPTPSGSMVTSESQLENKPYW |
| | | LQRAQGHNNGICWGNQLFVTVVDTTRSTNMTLCAEVKKESTYKNENEKE |
| | | YLRHGEEFDLQFIFQLCKITLTADVMTYIHKMDATILEDWQFGLTPPPS |
| | | ASLEDTYRFVTSTAITCQKNTPPKGKEDPLKDYMFWEVDLKEKFSADLD |
| | | QFPLGRKELLQAGLQARPKLKRPASSAPRTSTKKKKVKR |
| | 16 | MPVPVSKVVSTDEYVSRTSIYYYAGSSRLLTVGHPYFSIKNTSSGNGKK |
| | | VLVPKVSGLQYRVFRIKLPDPNKFGFPDTSFYNPETQRLVWACTGLEIG |
| | | RGQPLGVGISGHPLLNKEDDTETSNKYAGKPGIDNRECLSMDYKQTQLC |
| | | ILGCKPPIGEHWGKGTPCNNNSGNPGDCPPLQLINSVIQDGDMVDTGFG |
| | | CMDENTLQASKSDVPIDICSSVCKYPDYLQMASEPYGDSLFFELRREQM |
| | | FVRHFFNRAGTLGDPVPGDLYIQGSNSGNTATVQSSAFFPTPSGSMVTS |
| | | ESQLENKPYWLQRAQGHNNGICWGNQLFVTVVDTTRSTNMTLCAEVKKE |
| | | STYKNENFKEYLRHGEEFDLQFIFQLCKITLTADVMTYIHKMDATILED |
| | | WQFGLTPPPSASLEDTYRFVTSTAITCQKNTPPKGKEDPLKDYMFWEVD |
| | | LKEKFSADLDQFPLGRKELLQAGLQARPKLKRPASSAPRTSTKKKKVKR |
| | 26 | MSVWRPSEATVYLPPVPGSKVVSTDEYVSRTSIYYYAGSSRLLTVGHPY |
| | | FSIKNTSSGNGKKVLVPKVSGLQYRVFRIKLPDPNKFGFPDTSFYNPET |
| | | QRLVWACTGLEIGRGQPLGVGISGHPLLNKEDDTETSNKYAGKPGIDNR |
| | | ECLSMDYKQTQLCILGCKPPIGEHWGKGTPCNNNSGNPGDCPPLQLINS |
| | | VIQDGDMVDTGFGCMDENTLQASKSDVPIDICSSVCKYPDYLQMASEPY |
| | | GDSLFFFLRREQMFVRHFFNRAGTLGDPVPGDLYIQGSNSGNTATVQSS |
| | | AFFPTPSGSMVTSESQLENKPYWLQRAQGHNNGICWGNQLFVTVVDTTR |
| | | STNMTLCAEVKKESTYKNENFKEYLRHGEEFDLQFIFQLCKITLTADVM |
| | | TYIHKMDATILEDWQFGLTPPPSASLEDTYREVISTAITCQKNTPPKGK |
| | | EDPLKDYMFWEVDLKEKFSADLDQFPLGRKELLQAGLQARPKLKRPASS |
| | | APRTSTKKKKVKR |
| HPV58 | 17 | MVLILCCTLAILFCVADVNVFHIFLQMSVWRPSEATVYLPPVPVSKVVS |
| L1 | | TDEYVSRTSIYYYAGSSRLLAVGNPYFSIKSPNNNKKVLVPKVSGLQYR |
| | | VFRVRLPDPNKFGFPDTSFYNPDTQRLVWACVGLEIGRGQPLGVGVSGH |
| | | PYLNKFDDTETSNRYPAQPGSDNRECLSMDYKQTQLCLIGCKPPTGEHW |
| | | GKGVACNNNAAATDCPPLELENSIIEDGDMVDTGFGCMDEGTLQANKSD |
| | | VPIDICNSTCKYPDYLKMASEPYGDSLFFFLRREQMFVRHFFNRAGKLG |
| | | EAVPDDLYIKGSGNTAVIQSSAFFPTPSGSIVTSESQLENKPYWLQRAQ |
| | | GHNNGICWGNQLFVTVVDTTRSTNMTLCTEVTKEGTYKNDNFKEYVRHV |
| | | EEYDLQFVFQLCKITLTAEIMTYIHTMDSNILEDWQFGLTPPPSASLQD |
| | | TYRFVTSQAITCQKTAPPKEKEDPLNKYTFWEVNLKEKESADLDQFPLG |
| | | RKFLLQSGLKAKPRLKRSAPTTRAPSTKRKKVKK |
| | 18 | MTVYLPPVPVSKVVSTDEYVSRTSIYYYAGSSRLLAVGNPYESIKSPNN |
| | | NKKVLVPKVSGLQYRVERVRLPDPNKFGFPDTSFYNPDTQRLVWACVGL |
| | | EIGRGQPLGVGVSGHPYLNKEDDTETSNRYPAQPGSDNRECLSMDYKQT |
| | | QLCLIGCKPPTGEHWGKGVACNNNAAATDCPPLELENSIIEDGDMVDTG |
| | | FGCMDFGTLQANKSDVPIDICNSTCKYPDYLKMASEPYGDSLFFELRRE |
| | | QMFVRHFFNRAGKLGEAVPDDLYIKGSGNTAVIQSSAFFPTPSGSIVTS |
| | | ESQLENKPYWLQRAQGHNNGICWGNQLFVTVVDTTRSTNMTLCTEVTKE |
| | | GTYKNDNFKEYVRHVEEYDLQFVFQLCKITLTAEIMTYIHTMDSNILED |
| | | WQFGLTPPPSASLQDTYRFVTSQAITCQKTAPPKEKEDPLNKYTFWEVN |
| | | LKEKFSADLDQFPLGRKELLQSGLKAKPRLKRSAPTTRAPSTKRKKVKK |
| | 27 | MSVWRPSEATVYLPPVPVSKVVSTDEYVSRTSIYYYAGSSRLLAVGNPY |
| | | ESIKSPNNNKKVLVPKVSGLQYRVERVRLPDPNKFGFPDTSFYNPDTQR |
| | | LVWACVGLEIGRGQPLGVGVSGHPYENKEDDTETSNRYPAQPGSDNREC |
| | | LSMDYKQTQLCLIGCKPPTGEHWGKGVACNNNAAATDCPPLELENSIIE |
| | | DGDMVDTGFGCMDFGTLQANKSDVPIDICNSTCKYPDYLKMASEPYGDS |
| | | LFFFLRREQMFVRHFFNRAGKLGEAVPDDLYIKGSGNTAVIQSSAFFPT |
| | | PSGSMVTSESQLENKPYWLQRAQGHNNGICWGNQLFVTVVDTTRSTNMT |
| | | LCTEVTKEGTYKNDNFKEYVRHVEEYDLQFVFQLCKITLTAEIMTYIHT |
| | | MDSNILEDWQFGLTPPPSASLQDTYRFVTSQAITCQKTAPPKEKEDPLN |
| | | KYTFWEVNLKEKFSADLDQFPLGRKELLQSGLKAKPRLKRSAPTTRAPS |
| | | TKRKKVKK |
| HPV L1 | 30 | MALWRTSDSKVYLPPTPVSRVVNTDEYVTRTGIYYYAGSSRLLTLGHPYFSIPK |
| | | TGQKAEIPKVSAYQYRVFRVHLPDPNKFGLPDPQLYNPDTERLVWACVGVEVGR |
| | | GQPLGIGLSGHPLFNKLDDTENSHLATVNADTDNRDNVSVDNKQTQLCIIGCTP |
| | | PLGEHWGIGTICKNTQTQRGDCPPLELISSIIEDGDMIDTGFGAMDFTALQATK |
| | | SDVPIDISQSTCKYPDYLKMSADTYGNSMFFFLRREQLFARHFYNKAGAVGDAI |
| | | PTTLYIKGAESGREPPTSSIYSATPSGSMVTSDAQLENKPYWLQRAQGHNNGIC |
| | | WGNQLFVTCVDTTRSTNLTISTLSAASASTPFKPSDYKQFIRHGEEYELQFIFQ |
| | | LCKITLTTDVMAYIHLMNASILEDWNFGLTLPPTASLEDAYRFIKNSATTCQRN |
| | | APPVPKEDPFQKFKFWDVDLKEKFSIDLDQFPLGRKFMLQAGIQRRPKLGTKRP |
| | | LSSTSSSTKRKKRKLTK |
| HPV35 | 31 | MSLWRSNEATVYLPPVSVSKVVSTDEYVTRTNIYYHAGSSRLLAVGHPYYAIKK |
| L1 | | QDSNKIAVPKVSGLQYRVFRVKLPDPNKFGFPDTSFYDPASQRLVWACTGVEVG |
| | | RGQPLGVGISGHPLLNKLDDTENSNKYVGNSGTDNRECISMDYKQTQLCLIGCR |
| | | PPIGEHWGKGTPCNANQVKAGECPPLELLNTVLQDGDMVDTGFGAMDFTTLQAN |
| | | KSDVPLDICSSICKYPDYLKMVSEPYGDMLFFYLRREQMFVRHLFNRAGTVGET |
| | | VPADLYIKGTTGTLPSTSYFPTPSGSMVTSDAQIFNKPYWLQRAQGHNNGICWS |
| | | NQLFVTVVDTTRSTNMSVCSAVSTSDSTYKNDNFKEYLRHGEEYDLQFIFQLCK |
| | | ITLTADVMTYIHSMNPSILEDWNFGLTPPPSGTLEDTYRYVTSQAVTCQKPSAP |
| | | KPKDDPLKNYTFWEVDLKEKFSADLDQFPLGRKELLQAGLKARPNFRLGKRAAP |
| | | ASTSKKSSTKRRKVKS |
| HPV39 | 32 | MAMWRSSDSMVYLPPPSVAKVVNTDDYVTRTGIYYYAGSSRLLTVGHPYFKVGM |
| L1 | | NGGRKQDIPKVSAYQYRVFRVTLPDPNKFSIPDASLYNPETQRLVWACVGVEVG |
| | | RGQPLGVGISGHPLYNRQDDTENSPFSSTTNKDSRDNVSVDYKQTQLCIIGCVP |
| | | AIGEHWGKGKACKPNNVSTGDCPPLELVNTPIEDGDMIDTGYGAMDFGALQETK |
| | | SEVPLDICQSICKYPDYLQMSADVYGDSMFFCLRREQLFARHFWNRGGMVGDAI |
| | | PAQLYIKGTDIRANPGSSVYCPSPSGSMVTSDSQLFNKPYWLHKAQGHNNGICW |
| | | HNQLFLTVVDTTRSTNFTLSTSIESSIPSTYDPSKFKEYTRHVEEYDLQFIFQL |
| | | CTVTLTTDVMSYIHTMNSSILDNWNFAVAPPPSASLVDTYRYLQSAAITCQKDA |
| | | PAPEKKDPYDGLKFWNVDLREKFSLELDQFPLGRKFLLQARVRRRPTIGPRKRP |
| | | AASTSSSSATKHKRKRVSK |
| HPV51 | 33 | MALWRTNDSKVYLPPAPVSRIVNTEEYITRTGIYYYAGSSRLITLGHPYFPIPK |
| L1 | | TSTRAAIPKVSAFQYRVFRVQLPDPNKFGLPDPNLYNPDTDRLVWGCVGVEVGR |
| | | GQPLGVGLSGHPLENKYDDTENSRIANGNAQQDVRDNTSVDNKQTQLCIIGCAP |
| | | PIGEHWGIGTTCKNTPVPPGDCPPLELVSSVIQDGDMIDTGFGAMDFAALQATK |
| | | SDVPLDISQSVCKYPDYLKMSADTYGNSMFFHLRREQIFARHYYNKLGSVGEDI |
| | | PNDYYIKGSGNGRDPIESYIYSATPSGSMITSDSQIFNKPYWLHRAQGHNNGIC |
| | | WNNQLFITCVDTTRSTNLTISTATAAVSPTFTPSNFKQYIRHGEEYELQFIFQL |
| | | CKITLTTEVMAYLHTMDPTILEQWNFGLTLPPSASLEDAYRFVRNAATSCQKDT |
| | | PPQAKPDPLAKYKFWDVDLKERFSLDLDQFALGRKFLLQVGVQRKPRPGLKRPA |
| | | SSASSSSSSSAKRKRVKK |
| HPV53 | 34 | MAVWRPSDSKVYLPPTPVSKVITTDAYVKRTTIFYHAGSSRLLTVGHPYYPISK |
| L1 | | SGKTDIPKVSAFQYRVFRVRLPDPNKFGLPDTNIFNPDQERLVWACVGLEIGRG |
| | | QPLGVGVSGHPLFNRLDDTESSSIAIQDTAPDSRDNISVDPKQTQLCIIGCAPA |
| | | IGEHWTKGTACRSTPTTAGDCPPLELINSPIEDGDMVDTGFGALNFKALQESKS |
| | | DVPLDIVQSTCKYPDYLKMSADAYGDSMWFYLRREQLFTRHFFNRSGVIGEEIP |
| | | NDLYIKGSNDRDPPPSSVYVATPSGSMITSEAQLFNKPYWLQRAQGHNNGICWN |
| | | NQLFVTVVDTTRNTNMTLSATTQSMSTYNSKQIKQYVRHAEEYELQFVFQLCKI |
| | | SLSAEVMAYLHTMNSTLLEDWNIGLSPPVATSLEDKYRYVKSAAITCQKDQPPP |
| | | EKQDPLSKYKFWEVNLQNSFSADLDQFPLGRKFLMQVGVRTEPPVSSKKRSAPT |
| | | TSTSAPSSKRKRK |
| HPV56 | 35 | MATWRPSENKVYLPPTPVSKVVATDSYVKRTSIFYHAGSSRLLAVGHPYYSVTK |
| L1 | | DNTKTNIPKVSAYQYRVFRVRLPDPNKFGLPDTNIYNPDQERLVWACVGLEVGR |
| | | GQPLGAGLSGHPLFNRLDDTESSNLANNNVIEDSRDNISVDGKQTQLCIVGCTP |
| | | AMGEHWTKGAVCKSTQVTTGDCPPLALINTPIEDGDMIDTGFGAMDFKVLQESK |
| | | AEVPLDIVQSTCKYPDYLKMSADAYGDSMWFYLRREQLFARHYFNRAGKVGETI |
| | | PAELYLKGSNGREPPPSSVYVATPSGSMITSEAQLENKPYWLQRAQGHNNGICW |
| | | GNQLFVTVVDTTRSTNMTISTATEQLSKYDARKINQYLRHVEEYELQFVFQLCK |
| | | ITLSAEVMAYLHNMNANLLEDWNIGLSPPVATSLEDKYRYVRSTAITCQREQPP |
| | | TEKQDPLAKYKFWDVNLQDSFSTDLDQFPLGRKFLMQLGTRSKPAVATSKKRSA |
| | | PTSTSTPAKRKRR |
| HPV59 | 36 | MALWRSSDNKVYLPPPSVAKVVSTDDYVTRTSIFYHAGSSRLLTVGHPYFKVPK |
| L1 | | GGNGRQDVPKVSAYQYRVFRVNLPDPNKFGLPDNTVYDPNSQRLVWACVGVEIG |
| | | RGQPLGVGLSGHPFYNKLDDTENSHVASAVDNKDTRDNVSVDYKQTQLCIIGCV |
| | | PAIGEHWTKGTACKPNTVVQGDCPPLELINTPIEDGDMVDTGYGAMDFKLLQDT |
| | | KSEVPLDICQSICKYPDYLQMSADAYGDSMFFCLRREQVFARHFWNRSGTMGDQ |
| | | IPESLYIKGTDIRATPGSYLYSPSPSGSVVTSDSQLFNKPYWLHKAQGLNNGIC |
| | | WHNQLFLTVVDTTRSTNLSVCASTTSSIPNVYTPTSFKEYARHVEEFDLQFIFQ |
| | | LCKITLTTEVMSYIHNMNTTILEDWNFGVTPPPTASLVDTYRFVQSAAVTCQKD |
| | | TAPPVKQDPYDKLKFWTVDLKERFSADLDQFPLGRKFLLQLGARPKPTIGPRKR |
| | | AAPAPTSTPSPKRVKRRKSSRK |
| HPV66 | 37 | MAMWRPSDNKVYLPPTPVSKVVATDTYVKRTSIFYHAGSSRLLAVGHPYYSVSK |
| L1 | | SGTKTNIPKVSAYQYRVFRVRLPDPNKFGLPDPSFYNPDQERLVWACVGLEVGR |
| | | GQPLGAGLSGHPLFNRLDDTEVSNLAGNNVIEDSRDNISVDCKQTQLCIVGCAP |
| | | ALGEHWTKGAVCKSTPGNTGDCPPLALVNTPIEDGDMVDTGFGAMDFKLLQESK |
| | | AEVPLDIVQSTCKYPDYLKMSADAYGDSMWFYLRREQLFARHYFNRAGNVGEAI |
| | | PTDLYWKGGNGRDPPPSSVYVATPSGSMITSEAQLENKPYWLQRAQGHNNGICW |
| | | GNQVFVTVVDTTRSTNMTINAAKSTLTKYDAREINQYLRHVEEYELQFVFQLCK |
| | | ITLTAEVMAYLHNMNNTLLDDWNIGLSPPVATSLEDKYRYIKSTAITCQREQPP |
| | | AEKQDPLAKYKFWEVNLQDSFSADLDQFPLGRKFLMQLGPRPPRPKASVSASKR |
| | | RAAPTSSSSSPAKRKKR |
| HPV67 | 38 | MSVWRPSEATVYLPPVPVSKVVSTDEYVSRTSIYYYAGSSRLLAVGHPYFSIPN |
| L1 | | PSNTKKVLVPKVSGLQYRVFRVHLPDPNKFGFPDTSFYNPDTQRLVWACVGIEI |
| | | GRGQPLGVGISGHPLLNKFDDTETNNKYPSQPGTDNRECLSMDAKQTQLCIIGC |
| | | KPPTGEHWGKGTPCSGNSTGPGACPPLELMNTVIEDGDMIDTGFGCMDFKSLQA |
| | | NKSDVPLDICTSICKYPDYLGMASEAYGDSLFFFLRREQMFVRHLFNRAGKLGE |
| | | DVPTDLYFKGSANTSALQTSAFFPTPSGSMVSSESQLFNKPYWLQRAQGHNNGI |
| | | CWGNQIFVTVVDTTRSTNMTLCSEEKSEATYKNENFKEYLRHVEEYDLQFIFQL |
| | | CKISLTANVMQYIHTMNPDILEDWQFGLTPPPSGNLQDTYRFVTSQAITCQKTA |
| | | TPTAKEDPLKKYSFWEINLKEKFSADLDQFPLGRKFLLQAGFTAKPKLKRSSPS |
| | | SSSSSSAKRKKVKR |
| HPV68 | 39 | MALWRSSDNMVYLPPPSVAKVVNTDDYVTRTGIYYYAGTSRLLTVGHPYFKVPM |
| L1 | | SGGRKQDIPKVSAYQYRVFRVSLPDPNKFSLPESTLYNPDTQRLVWACVGVEIG |
| | | RGQPLGVGLSGHPLYNRLDDTENSPFSSNKNPKDSRDNVSVDYKQTQLCIIGCV |
| | | PAIGEHWAKGKSCKPSNVQPGDCPPLELVNTPIQDGDMIDTGYGAMDFSTLQET |
| | | KSEVPLDICQSVCKYPDYLQMSADVYGDSMFFCLRRDQLFARHFWNRGGMVGDT |
| | | IPTELYIKGTDIRDSPSSYVYAPSPSGSMVSSDSQLFNKPNWLHKAQGHNNGIC |
| | | WHNQLFLTVVDTTRSTNFTLSTTTESAVPNIYDPNKFKEYVRHVEEYDLQFIFQ |
| | | LCTITLSTDVMSYIHTMNPAILDDWNFGVAPPPSASLVDTYRYLQSAAITCQKD |
| | | APAPTKKDPYDGLNFWNVNLKEKFSSELDQFPLGRKFLLQAGVRRRPTIGPRKR |
| | | PATAPTASTSKHKRKRVSK |
| HPV70 | 40 | MALWRSSDNTVYLPPPSVAKVVNTDDYVTRTGIYYYAGSSRLLTVGHPYFKVPV |
| L1 | | NGGRKQEIPKVSAYQYRVFRVSLPDPNKFGLPDPSLYNPDTQRLVWACIGVEIG |
| | | RGQPLGVGVSGHPLYNRLDDTENSHESSAVNTQDSRDNVSVDYKQTQLCIIGCV |
| | | PAMGEHWAKGKACKSTTVQQGDCPPLELVNTAIEDGDMIDTGYGAMDFRTLQET |
| | | KSEVPLDICQSVCKYPDYLQMSADVYGDSMFFCLRKEQLFARHFWNRGGMVGDT |
| | | IPSELYIKGTDIRDRPGTHVYSPSPSGSMVSSDSQLFNKPYWLHKAQGHNNGIC |
| | | WHNQLFITVVDTTRSTNFTLSACTETAIPAVYSPTKFKEYTRHVEEYDLQFIFQ |
| | | LCTITLTADVMAYIHTMNPAILDNWNIGVTPPPSASLVDTYRYLQSAAIACQKD |
| | | APAPEKKDPYDDLKFWNVDLKEKFSTELDQFPLGRKFLLQVGARRRPTIGPRKR |
| | | PASAKSSSSASKHKRKRVSK |
| HPV73 | 41 | MWRPTDAKVYLPPVSVSKVVSTDEYVTRTNIYYYAGSTRLLAVGHPYFPIKDSQ |
| L1 | | KRKTIVPKVSGLQYRVFRLRLPDPNKFGFPDASFYNPDKERLVWACSGVEVGRG |
| | | QPLGIGTSGNPFMNKLDDTENAPKYIAGQNTDGRECMSVDYKQTQLCILGCRPP |
| | | LGEHWGPGTPCTSQTVNTGDCPPLELKNTPIQDGDMIDVGFGAMDEKALQANKS |
| | | DVPIDISNTTCKYPDYLGMAADPYGDSMWFYLRREQMFVRHLFNRAGDTGDKIP |
| | | DDLMIKGTGNTATPSSCVFYPTPSGSMVSSDAQLFNKPYWLQKAQGQNNGICWH |
| | | NQLFLTVVDTTRSTNFSVCVGTQASSSTTTYANSNFKEYLRHAEEFDLQFVFQL |
| | | CKISLTTEVMTYIHSMNSTILEEWNFGLTPPPSGTLEETYRYVTSQAISCQRPQ |
| | | PPKETEDPYAKLSFWDVDLKEKFSAELDQFPLGRKELLQLGMRARPKLQASKRS |
| | | ASATTSATPKKKRAKRI |
| HPV82 | 42 | MALWRTNDSKVYLPPAPVSRIVNTEEYITRTGIYYYAGSSRLITLGHPYFSIPK |
| L1 | | TNTRAEIPKVSAFQYRVFRVQLPDPNKFGLPDPNLENPDTDRLVWGCVGVEVGR |
| | | GQPLGVGLSGHPLENKYDDTENSRFANGNDQQDVRDNISVDNKQTQLCIIGCAP |
| | | PIGEHWATGTTCKNVPVPQGDCPPLELVSTVIEDGDMVDTGFGAMDFANLQATK |
| | | SDVPLDIAQSVCKYPDYLKMSADTYGNSMFFHLRREQIFARHYYNKAGVVGDAI |
| | | PDKAYIKGTGAGRDPISSYIYSATPSGSMITSDSQIFNKPYWLHRAQGHNNGIC |
| | | WNNQLFITCVDTTKSTNLTISTAVTPSVAQTFTPANFKQYIRHGEEYELQFIFQ |
| | | LCKITLTTEIMAYLHTMDSTILEQWNFGLTLPPSASLEDAYRFVKNAATSCHKD |
| | | SPPQAKEDPLAKYKFWNVDLKERFSLDLDQFALGRKELLQIGAQRKPRPGLKRP |
| | | APSSSASSSAKRKRVKK |

In a preferred embodiment, the one or more HPV antigen(s) are HPV L1 proteins or immunogenic fragments thereof.

As used herein, an “immunogenic fragment” refers to a fragment of a reference antigen containing one or more epitopes (e.g., linear, conformational or both) capable of stimulating a host's immune system to make a humoral and/or cellular antigen-specific immunological response (i.e. an immune response which specifically recognizes a naturally occurring polypeptide, e.g., a viral or bacterial protein). An “epitope” is that portion of an antigen that determines its immunological specificity. T-and B-cell epitopes can be identified empirically (e.g. using PEPSCAN or similar methods). In a preferred embodiment, the immunogenic fragment induces an immune response, suitably a humoral or Tcell response, which is similar to the immune response induced by the reference antigen. Suitably, the immunogenic fragment induces a humoral or T cell response in mice which is not more than 10-fold lower, more suitably not more than 5-fold lower, not more than 2-fold lower or not lower, than the immune response induced by the reference antigen.

As used herein, an “immunogenic fragment of a HPV L1 protein” refers to a fragment of a naturally-occurring HPV L1 protein of at least 50, 60, 100, 200, 300, 400, 450 or more amino acids, or a peptide having an amino acid sequence of at least 60%, 65%, 70%, 75%, 80%, 85%, 90%, 95%, 97%, 98%, 99%, or 99.5% sequence identity to a naturally-occurring HPV L1 protein (or to a fragment of a naturally-occurring HPV L1 protein of at least about 50, 60, 100, 200, 300, 400, 450 or more amino acids). Thus, an immunogenic fragment of a HPV L1 protein may be a fragment of a naturally occurring HPV L1 protein, of at least 50 amino acids, and may comprise one or more amino acid substitutions, deletions or additions. Any of the HPV L1 protein immunogenic fragments may additionally comprise an initial methionine residue where required.

In one embodiment, the one or more HPV antigen(s) form VLPs. A “virus like particle” (VLP) is a self-assemblage of structural capsid proteins that mimics a virus but does not comprise the genetic material of a virus. A VLP is therefore not infectious. Suitably, the one or more HPV antigen(s) form VLPs comprising HPV L1, L2, chimeric L1 and/or chimeric L1/L2 proteins or immunogenic fragments thereof. In a preferred embodiment, the one or more HPV antigen(s) form VLPs consisting of HPV L1 proteins or immunogenic fragments thereof.

HPV VPLs and methods for the production of VLPs are well known in the art. VLPs typically are produced recombinantly from the HPV L1 protein of the virus and can also include the L2 protein. See for example WO9420137, U.S. Pat. No. 5,985,610, WO9611272, EP595935 for VLPs. Suitable expression systems for HPV VLPs, in particular L1 VLPs include without limitation, any prokaryotic and/or eukaryotic system(s) including baculoviruses, adenoviruses, SV40, E. coli, CHO cells, vaccinia virus, insect viruses, yeast, bacteriophage virus or modified viruses, agrobacteria, DNA plasmids, vectors and the like. The host cell for expression of the L1 coding sequence is dependent on the expression system used. Examples of suitable host cells include, without limitation, bacteria (such as E. coli), microorganisms such as yeast (such as Saccharomyces cerevisiae), mammalian cells (eukaryotic) and insect cells. Methods for producing HPV VLPs in E. coli are disclosed in China patent No: ZL200610140613.0 and in Pan H, et al., 2017¹⁵, in Gu, Y. et al., 2017¹⁶, in Wang D., et al., 2017¹⁷ and in Wei M. et al. 2018¹⁸. When using the baculovirus expression system, insect cells, such as Sf-9 or Sf-21 are preferred. HPV VLPs can also be produced in plants such as tobacco plants using recombinant Agrobacterium constructs (see eg. Naupu, P. N. et al, 2020¹²).

In one embodiment, the HPV L1 VLPS are produced in E. coli, yeast cells or in a baculovirus expression system. In a preferred embodiment, the HPV L1 VLPS are produced in E. coli.

In one embodiment, the one or more HPV antigen(s) comprise at least two, for example at least three, at least four, at least five, at least six, at least seven, at least eight, at least nine HPV antigen.

In a preferred embodiment, the one or more HPV antigen(s) comprise at least nine HPV antigens.

In one embodiment, the one or more HPV antigen(s) are from HPV types selected from HPV types 6, 11, 16, 18, 26, 31, 33, 35, 39, 45, 51, 52, 53, 56, 58, 59, 67, 68, 70, 73 and 82.

In one embodiment, the one or more HPV antigen(s) are from HPV types selected from HPV types 6, 11, 16, 18, 31, 33, 35, 39, 45, 52, 58 and 59.

In a preferred embodiment, the one or more HPV antigen(s) are from HPV types selected from HPV types 6, 11, 16, 18, 31, 33, 45, 52 and 58.

In one embodiment, the one or more HPV antigen(s) comprise HPV antigen(s) from HPV types 16 and 18.

In one embodiment, the one or more HPV antigen(s) comprise HPV antigen(s) from HPV types 6, 11, 16, and 18.

In a preferred embodiment, the one or more HPV antigen(s) comprise or consist of HPV antigen(s) from HPV types 6, 11, 16, 18, 31, 33, 45, 52 and 58.

In one embodiment, the one or more HPV antigen(s) comprise HPV antigen(s) from HPV types 6, 11, 16, 18, 31, 33, 45, 52 and 58, and optionally HPV antigen(s) from HPV types 35, 39 and/or 59.

In one embodiment, the one or more HPV antigen(s) comprise L1 VLPs from HPV types 16 and 18.

In one embodiment, the one or more HPV antigen(s) comprise L1 VLPs from HPV types 6, 11, 16, and 18.

In one embodiment, the one or more HPV antigen(s) comprise L1 VLPs from HPV types 6, 11, 16, 18, 31, 33, 45, 52 and 58 L1 VLPs, and optionally from HPV types 35, 39 and/or 59.

In one embodiment, the one or more HPV antigen(s) comprise a HPV 6 L1 protein comprising or consisting of an amino-acid sequence which is at least 80% identical to SEQ ID NO: 1, suitably at least 90%, at least 95%, 99% or 100% identical to SEQ ID NO: 1.

The sequence of the HPV 6 L1 protein may be C-terminally or N-terminally truncated compared to the corresponding naturally occurring HPV 6 L1 protein. Suitably, the HPV 6 L1 protein comprises a C-terminal truncation or N-terminal truncation of 50 amino acids or less compared to the corresponding naturally occurring HPV 6 L1 protein, for example 40 amino acids or less, 30 amino acids or less, 20 amino acids or less, 15 amino acids or less, 10 amino acids or less, 5 amino acids or less. In one embodiment, the HPV 6 L1 protein comprises a C-terminal truncation of 50 amino acids or less compared to SEQ ID NO: 1 or to a variant thereof, for example 40 amino acids or less, 30 amino acids or less, 20 amino acids or less, 15 amino acids or less, 10 amino acids or less, 5 amino acids or less. In one embodiment, the HPV 6 L1 protein comprises a N-terminal truncation of 50 amino acids or less compared to SEQ ID NO: 1 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 6 L1 protein comprising or consisting of the amino acid sequence SEQ ID NO: 2 or a variant thereof.

In one embodiment, the one or more HPV antigen(s) comprise a HPV 6 L1 protein comprising or consisting of the amino acid sequence SEQ ID NO: 19 or a variant thereof.

In one embodiment, the one or more HPV antigen(s) comprise a HPV 11 L1 protein comprising or consisting of an amino-acid sequence which is at least 80% identical to SEQ ID NO: 3, suitably at least 90%, at least 95%, 99% or 100% identical to SEQ ID NO: 3.

The sequence of the HPV 11 L1 protein may be C-terminally or N-terminally truncated compared to the corresponding naturally occurring HPV 11 L1 protein. Suitably, the HPV 11 L1 protein comprises a C-terminal truncation or N-terminal truncation of 50 amino acids or less compared to the corresponding naturally occurring HPV 11 L1 protein, for example 40 amino acids or less, 30 amino acids or less, 20 amino acids or less, 15 amino acids or less, 10 amino acids or less, 5 amino acids or less. In one embodiment, the HPV 11 L1 protein comprises a C-terminal truncation of 50 amino acids or less compared to SEQ ID NO: 3 or to a variant thereof, for example 40 amino acids or less, 30 amino acids or less, 20 amino acids or less, 15 amino acids or less, 10 amino acids or less, 5 amino acids or less. In one embodiment, the HPV 11 L1 protein comprises a N-terminal truncation of 50 amino acids or less compared to SEQ ID NO: 3 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 11 L1 protein comprising or consisting of the amino acid sequence SEQ ID NO: 4 or a variant thereof.

In one embodiment, the one or more HPV antigen(s) comprise a HPV 11 L1 protein comprising or consisting of the amino acid sequence SEQ ID NO: 20 or a variant thereof.

In one embodiment, the one or more HPV antigen(s) comprise a HPV 16 11 protein comprising or consisting of an amino-acid sequence which is at least 80% identical to SEQ ID NO: 5, suitably at least 90%, at least 95%, 99% or 100% identical to SEQ ID NO: 5.

The sequence of the HPV 16 L1 protein may be C-terminally or N-terminally truncated compared to the corresponding naturally occurring HPV 16 L1 protein. Suitably, the HPV 16 L1 protein comprises a C-terminal truncation or N-terminal truncation of 50 amino acids or less compared to the corresponding naturally occurring HPV 16 L1 protein, for example 40 amino acids or less, 30 amino acids or less, 20 amino acids or less, 15 amino acids or less, 10 amino acids or less, 5 amino acids or less. In one embodiment, the HPV 16 L1 protein comprises a C-terminal truncation of 50 amino acids or less compared to SEQ ID NO: 5 or to a variant thereof, for example 40 amino acids or less, 30 amino acids or less, 20 amino acids or less, 15 amino acids or less, 10 amino acids or less, 5 amino acids or less. In one embodiment, the HPV 16 L1 protein comprises a N-terminal truncation of 50 amino acids or less compared to SEQ ID NO: 5 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 16 L1 protein comprising or consisting of the amino acid sequence SEQ ID NO: 6 or a variant thereof.

In one embodiment, the one or more HPV antigen(s) comprise a HPV 16 L1 protein comprising or consisting of the amino acid sequence SEQ ID NO: 21 or a variant thereof.

In one embodiment, the one or more HPV antigen(s) comprise a HPV 16 L1 protein comprising or consisting of the amino acid sequence SEQ ID NO: 28 or a variant thereof.

In one embodiment, the one or more HPV antigen(s) comprise a HPV 18 11 protein comprising or consisting of an amino-acid sequence which is at least 80% identical to SEQ ID NO: 7, suitably at least 90%, at least 95%, 99% or 100% identical to SEQ ID NO: 7.

The sequence of the HPV 18 L1 protein may be C-terminally or N-terminally truncated compared to the corresponding naturally occurring HPV 18 L1 protein. Suitably, the HPV 18 L1 protein comprises a C-terminal truncation or N-terminal truncation of 50 amino acids or less compared to the corresponding naturally occurring HPV 18 L1 protein, for example 40 amino acids or less, 30 amino acids or less, 20 amino acids or less, 15 amino acids or less, 10 amino acids or less, 5 amino acids or less. In one embodiment, the HPV 18 L1 protein comprises a C-terminal truncation of 50 amino acids or less compared to SEQ ID NO: 7 or to a variant thereof, for example 40 amino acids or less, 30 amino acids or less, 20 amino acids or less, 15 amino acids or less, 10 amino acids or less, 5 amino acids or less. In one embodiment, the HPV 18 L1 protein comprises a N-terminal truncation of 50 amino acids or less compared to SEQ ID NO: 7 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 18 L1 protein comprising or consisting of the amino acid sequence SEQ ID NO: 8 or a variant thereof.

In one embodiment, the one or more HPV antigen(s) comprise a HPV 18 L1 protein comprising or consisting of the amino acid sequence SEQ ID NO: 22, or a variant thereof.

In one embodiment, the one or more HPV antigen(s) comprise a HPV 18 L1 protein comprising or consisting of the amino acid sequence SEQ ID NO: 29, or a variant thereof.

In one embodiment, the one or more HPV antigen(s) comprise a HPV 31 L1 protein comprising or consisting of an amino-acid sequence which is at least 80% identical to SEQ ID NO: 9, suitably at least 90%, at least 95%, 99% or 100% identical to SEQ ID NO: 9.

The sequence of the HPV 31 L1 protein may be C-terminally or N-terminally truncated compared to the corresponding naturally occurring HPV 31 L1 protein. Suitably, the HPV 31 L1 protein comprises a C-terminal truncation or N-terminal truncation of 50 amino acids or less compared to the corresponding naturally occurring HPV 31 L1 protein, for example 40 amino acids or less, 30 amino acids or less, 20 amino acids or less, 15 amino acids or less, 10 amino acids or less, 5 amino acids or less. In one embodiment, the HPV 31 L1 protein comprises a C-terminal truncation of 50 amino acids or less compared to SEQ ID NO: 9 or to a variant thereof, for example 40 amino acids or less, 30 amino acids or less, 20 amino acids or less, 15 amino acids or less, 10 amino acids or less, 5 amino acids or less. In one embodiment, the HPV 31 L1 protein comprises a N-terminal truncation of 50 amino acids or less compared to SEQ ID NO: 9 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 31 L1 protein comprising or consisting of the amino acid sequence SEQ ID NO: 10 or a variant thereof.

In one embodiment, the one or more HPV antigen(s) comprise a HPV 31 L1 protein comprising or consisting of the amino acid sequence SEQ ID NO: 23 or a variant thereof.

In one embodiment, the one or more HPV antigen(s) comprise a HPV 33 L1 protein comprising or consisting of an amino-acid sequence which is at least 80% identical to SEQ ID NO: 11, suitably at least 90%, at least 95%, 99% or 100% identical to SEQ ID NO: 11.

The sequence of the HPV 33 L1 protein may be C-terminally or N-terminally truncated compared to the corresponding naturally occurring HPV 33 L1 protein. Suitably, the HPV 33 L1 protein comprises a C-terminal truncation or N-terminal truncation of 50 amino acids or less compared to the corresponding naturally occurring HPV 33 L1 protein, for example 40 amino acids or less, 30 amino acids or less, 20 amino acids or less, 15 amino acids or less, 10 amino acids or less, 5 amino acids or less. In one embodiment, the HPV 33 L1 protein comprises a C-terminal truncation of 50 amino acids or less compared to SEQ ID NO: 11 or to a variant thereof, for example 40 amino acids or less, 30 amino acids or less, 20 amino acids or less, 15 amino acids or less, 10 amino acids or less, 5 amino acids or less. In one embodiment, the HPV 33 L1 protein comprises a N-terminal truncation of 50 amino acids or less compared to SEQ ID NO: 11 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 33 L1 protein comprising or consisting of the amino acid sequence SEQ ID NO: 12 or a variant thereof.

In one embodiment, the one or more HPV antigen(s) comprise a HPV 33 L1 protein comprising or consisting of the amino acid sequence SEQ ID NO: 24 or a variant thereof.

In one embodiment, the one or more HPV antigen(s) comprise a HPV 45 L1 protein comprising or consisting of an amino-acid sequence which is at least 80% identical to SEQ ID NO: 13, suitably at least 90%, at least 95%, 99% or 100% identical to SEQ ID NO: 13.

The sequence of the HPV 45 L1 protein may be C-terminally or N-terminally truncated compared to the corresponding naturally occurring HPV 45 L1 protein. Suitably, the HPV 45 L1 protein comprises a C-terminal truncation or N-terminal truncation of 50 amino acids or less compared to the corresponding naturally occurring HPV 45 L1 protein, for example 40 amino acids or less, 30 amino acids or less, 20 amino acids or less, 15 amino acids or less, 10 amino acids or less, 5 amino acids or less. In one embodiment, the HPV 45 L1 protein comprises a C-terminal truncation of 50 amino acids or less compared to SEQ ID NO: 13 or to a variant thereof, for example 40 amino acids or less, 30 amino acids or less, 20 amino acids or less, 15 amino acids or less, 10 amino acids or less, 5 amino acids or less. In one embodiment, the HPV 45 L1 protein comprises a N-terminal truncation of 50 amino acids or less compared to SEQ ID NO: 13 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 45 L1 protein comprising or consisting of the amino acid sequence SEQ ID NO: 14 or a variant thereof.

In one embodiment, the one or more HPV antigen(s) comprise a HPV 45 L1 protein comprising or consisting of the amino acid sequence SEQ ID NO: 25 or a variant thereof.

In one embodiment, the one or more HPV antigen(s) comprise a HPV 52 L1 protein comprising or consisting of an amino-acid sequence which is at least 80% identical to SEQ ID NO: 15, suitably at least 90%, at least 95%, 99% or 100% identical to SEQ ID NO: 15.

The sequence of the HPV 52 L1 protein may be C-terminally or N-terminally truncated compared to the corresponding naturally occurring HPV 52 L1 protein. Suitably, the HPV 52 L1 protein comprises a C-terminal truncation or N-terminal truncation of 50 amino acids or less compared to the corresponding naturally occurring HPV 52 L1 protein, for example 40 amino acids or less, 30 amino acids or less, 20 amino acids or less, 15 amino acids or less, 10 amino acids or less, 5 amino acids or less. In one embodiment, the HPV 52 L1 protein comprises a C-terminal truncation of 50 amino acids or less compared to SEQ ID NO: 15 or to a variant thereof, for example 40 amino acids or less, 30 amino acids or less, 20 amino acids or less, 15 amino acids or less, 10 amino acids or less, 5 amino acids or less. In one embodiment, the HPV 52 L1 protein comprises a N-terminal truncation of 50 amino acids or less compared to SEQ ID NO: 15 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 52 L1 protein comprising or consisting of the amino acid sequence SEQ ID NO: 16 or a variant thereof.

In one embodiment, the one or more HPV antigen(s) comprise a HPV 52 L1 protein comprising or consisting of the amino acid sequence SEQ ID NO: 26 or a variant thereof.

In one embodiment, the one or more HPV antigen(s) comprise a HPV 58 L1 protein comprising or consisting of an amino-acid sequence which is at least 80% identical to SEQ ID NO: 17, suitably at least 90%, at least 95%, 99% or 100% identical to SEQ ID NO: 17.

The sequence of the HPV 58 L1 protein may be C-terminally or N-terminally truncated compared to the corresponding naturally occurring HPV 58 L1 protein. Suitably, the HPV 58 L1 protein comprises a C-terminal truncation or N-terminal truncation of 50 amino acids or less compared to the corresponding naturally occurring HPV 58 L1 protein, for example 40 amino acids or less, 30 amino acids or less, 20 amino acids or less, 15 amino acids or less, 10 amino acids or less, 5 amino acids or less. In one embodiment, the HPV 58 L1 protein comprises a C-terminal truncation of 50 amino acids or less compared to SEQ ID NO: 17 or to a variant thereof, for example 40 amino acids or less, 30 amino acids or less, 20 amino acids or less, 15 amino acids or less, 10 amino acids or less, 5 amino acids or less. In one embodiment, the HPV 58 L1 protein comprises a N-terminal truncation of 50 amino acids or less compared to SEQ ID NO: 17 or to a variant thereof, for example 40 amino acids or less, 30 amino acids or less, 20 amino acids or less, 15 amino acids or less, 10 amino acids or less, 5 amino acids or less.

In a preferred embodiment, the one or more HPV antigen(s) comprise a HPV 58 L1 protein comprising or consisting of the amino acid sequence SEQ ID NO: 18 or a variant thereof.

In one embodiment, the one or more HPV antigen(s) comprise a HPV 58 L1 protein comprising or consisting of the amino acid sequence SEQ ID NO: 27 or a variant thereof.

In one embodiment, the one or more HPV antigen(s) comprise a HPV 35 L1 protein comprising or consisting of an amino-acid sequence which is at least 80% identical to SEQ ID NO: 31, suitably at least 90%, at least 95%, 99% or 100% identical to SEQ ID NO: 31.

The sequence of the HPV 35 L1 protein may be C-terminally or N-terminally truncated compared to the corresponding naturally occurring HPV 35 L1 protein. Suitably, the HPV 35 L1 protein comprises a C-terminal truncation or N-terminal truncation of 50 amino acids or less compared to the corresponding naturally occurring HPV 35 L1 protein, for example 40 amino acids or less, 30 amino acids or less, 20 amino acids or less, 15 amino acids or less, 10 amino acids or less, 5 amino acids or less. In one embodiment, the HPV 35 L1 protein comprises a C-terminal truncation of 50 amino acids or less compared to SEQ ID NO: 31 or to a variant thereof, for example 40 amino acids or less, 30 amino acids or less, 20 amino acids or less, 15 amino acids or less, 10 amino acids or less, 5 amino acids or less. In one embodiment, the HPV 35 L1 protein comprises a N-terminal truncation of 50 amino acids or less compared to SEQ ID NO: 31 or to a variant thereof, for example 40 amino acids or less, 30 amino acids or less, 20 amino acids or less, 15 amino acids or less, 10 amino acids or less, 5 amino acids or less.

In one embodiment, the one or more HPV antigen(s) comprise a HPV 39 L1 protein comprising or consisting of an amino-acid sequence which is at least 80% identical to SEQ ID NO: 32, suitably at least 90%, at least 95%, 99% or 100% identical to SEQ ID NO: 32.

The sequence of the HPV 39 L1 protein may be C-terminally or N-terminally truncated compared to the corresponding naturally occurring HPV 39 L1 protein. Suitably, the HPV 39 L1 protein comprises a C-terminal truncation or N-terminal truncation of 50 amino acids or less compared to the corresponding naturally occurring HPV 39 L1 protein, for example 40 amino acids or less, 30 amino acids or less, 20 amino acids or less, 15 amino acids or less, 10 amino acids or less, 5 amino acids or less. In one embodiment, the HPV 39 L1 protein comprises a C-terminal truncation of 50 amino acids or less compared to SEQ ID NO: 32 or to a variant thereof, for example 40 amino acids or less, 30 amino acids or less, 20 amino acids or less, 15 amino acids or less, 10 amino acids or less, 5 amino acids or less. In one embodiment, the HPV 39 L1 protein comprises a N-terminal truncation of 50 amino acids or less compared to SEQ ID NO: 32 or to a variant thereof, for example 40 amino acids or less, 30 amino acids or less, 20 amino acids or less, 15 amino acids or less, 10 amino acids or less, 5 amino acids or less.

In one embodiment, the one or more HPV antigen(s) comprise a HPV 59 L1 protein comprising or consisting of an amino-acid sequence which is at least 80% identical to SEQ ID NO: 36, suitably at least 90%, at least 95%, 99% or 100% identical to SEQ ID NO: 36.

The sequence of the HPV 59 L1 protein may be C-terminally or N-terminally truncated compared to the corresponding naturally occurring HPV 59 L1 protein. Suitably, the HPV 59 L1 protein comprises a C-terminal truncation or N-terminal truncation of 50 amino acids or less compared to the corresponding naturally occurring HPV 59 L1 protein, for example 40 amino acids or less, 30 amino acids or less, 20 amino acids or less, 15 amino acids or less, 10 amino acids or less, 5 amino acids or less. In one embodiment, the HPV 59 L1 protein comprises a C-terminal truncation of 50 amino acids or less compared to SEQ ID NO: 36 or to a variant thereof, for example 40 amino acids or less, 30 amino acids or less, 20 amino acids or less, 15 amino acids or less, 10 amino acids or less, 5 amino acids or less. In one embodiment, the HPV 59 L1 protein comprises a N-terminal truncation of 50 amino acids or less compared to SEQ ID NO: 36 or to a variant thereof, for example 40 amino acids or less, 30 amino acids or less, 20 amino acids or less, 15 amino acids or less, 10 amino acids or less,

In one embodiment, the one or more HPV antigen(s) comprise a HPV 26 L1 protein comprising or consisting of an amino-acid sequence which is at least 80% identical to SEQ ID NO: 30, suitably at least 90%, at least 95%, 99% or 100% identical to SEQ ID NO: 30.

The sequence of the HPV 26 L1 protein may be C-terminally or N-terminally truncated compared to the corresponding naturally occurring HPV 26 L1 protein. Suitably, the HPV 26 L1 protein comprises a C-terminal truncation or N-terminal truncation of 50 amino acids or less compared to the corresponding naturally occurring HPV 26 L1 protein, for example 40 amino acids or less, 30 amino acids or less, 20 amino acids or less, 15 amino acids or less, 10 amino acids or less, 5 amino acids or less. In one embodiment, the HPV 26 L1 protein comprises a C-terminal truncation of 50 amino acids or less compared to SEQ ID NO: 30 or to a variant thereof, for example 40 amino acids or less, 30 amino acids or less, 20 amino acids or less, 15 amino acids or less, 10 amino acids or less, 5 amino acids or less. In one embodiment, the HPV 26 L1 protein comprises a N-terminal truncation of 50 amino acids or less compared to SEQ ID NO: 30 or to a variant thereof, for example 40 amino acids or less, 30 amino acids or less, 20 amino acids or less, 15 amino acids or less, 10 amino acids or less, 5 amino acids or less.

In one embodiment, the one or more HPV antigen(s) comprise a HPV 39 L1 protein comprising or consisting of an amino-acid sequence which is at least 80% identical to SEQ ID NO: 32, suitably at least 90%, at least 95%, 99% or 100% identical to SEQ ID NO: 32.

The sequence of the HPV 39 L1 protein may be C-terminally or N-terminally truncated compared to the corresponding naturally occurring HPV 39 L1 protein. Suitably, the HPV 39 L1 protein comprises a C-terminal truncation or N-terminal truncation of 50 amino acids or less compared to the corresponding naturally occurring HPV 39 L1 protein, for example 40 amino acids or less, 30 amino acids or less, 20 amino acids or less, 15 amino acids or less, 10 amino acids or less, 5 amino acids or less. In one embodiment, the HPV 39 L1 protein comprises a C-terminal truncation of 50 amino acids or less compared to SEQ ID NO: 32 or to a variant thereof, for example 40 amino acids or less, 30 amino acids or less, 20 amino acids or less, 15 amino acids or less, 10 amino acids or less, 5 amino acids or less. In one embodiment, the HPV 39 L1 protein comprises a N-terminal truncation of 50 amino acids or less compared to SEQ ID NO: 32 or to a variant thereof, for example 40 amino acids or less, 30 amino acids or less, 20 amino acids or less, 15 amino acids or less, 10 amino acids or less,

In one embodiment, the one or more HPV antigen(s) comprise a HPV 51 L1 protein comprising or consisting of an amino-acid sequence which is at least 80% identical to SEQ ID NO: 33, suitably at least 90%, at least 95%, 99% or 100% identical to SEQ ID NO: 33.

The sequence of the HPV 51 L1 protein may be C-terminally or N-terminally truncated compared to the corresponding naturally occurring HPV 51 L1 protein. Suitably, the HPV 51 L1 protein comprises a C-terminal truncation or N-terminal truncation of 50 amino acids or less compared to the corresponding naturally occurring HPV 51 L1 protein, for example 40 amino acids or less, 30 amino acids or less, 20 amino acids or less, 15 amino acids or less, 10 amino acids or less, 5 amino acids or less. In one embodiment, the HPV 51 L1 protein comprises a C-terminal truncation of 50 amino acids or less compared to SEQ ID NO: 33 or to a variant thereof, for example 40 amino acids or less, 30 amino acids or less, 20 amino acids or less, 15 amino acids or less, 10 amino acids or less, 5 amino acids or less. In one embodiment, the HPV 51 L1 protein comprises a N-terminal truncation of 50 amino acids or less compared to SEQ ID NO: 33 or to a variant thereof, for example 40 amino acids or less, 30 amino acids or less, 20 amino acids or less, 15 amino acids or less, 10 amino acids or less, 5 amino acids or less.

In one embodiment, the one or more HPV antigen(s) comprise a HPV 53 L1 protein comprising or consisting of an amino-acid sequence which is at least 80% identical to SEQ ID NO: 34, suitably at least 90%, at least 95%, 99% or 100% identical to SEQ ID NO: 34.

The sequence of the HPV 53 L1 protein may be C-terminally or N-terminally truncated compared to the corresponding naturally occurring HPV 53 L1 protein. Suitably, the HPV 53 L1 protein comprises a C-terminal truncation or N-terminal truncation of 50 amino acids or less compared to the corresponding naturally occurring HPV 53 L1 protein, for example 40 amino acids or less, 30 amino acids or less, 20 amino acids or less, 15 amino acids or less, 10 amino acids or less, 5 amino acids or less. In one embodiment, the HPV 53 L1 protein comprises a C-terminal truncation of 50 amino acids or less compared to SEQ ID NO: 34 or to a variant thereof, for example 40 amino acids or less, 30 amino acids or less, 20 amino acids or less, 15 amino acids or less, 10 amino acids or less, 5 amino acids or less. In one embodiment, the HPV 53 L1 protein comprises a N-terminal truncation of 50 amino acids or less compared to SEQ ID NO: 34 or to a variant thereof, for example 40 amino acids or less, 30 amino acids or less, 20 amino acids or less, 15 amino acids or less, 10 amino acids or less,

In one embodiment, the one or more HPV antigen(s) comprise a HPV 56 L1 protein comprising or consisting of an amino-acid sequence which is at least 80% identical to SEQ ID NO: 35, suitably at least 90%, at least 95%, 99% or 100% identical to SEQ ID NO: 35.

The sequence of the HPV 56 L1 protein may be C-terminally or N-terminally truncated compared to the corresponding naturally occurring HPV 56 L1 protein. Suitably, the HPV 56 L1 protein comprises a C-terminal truncation or N-terminal truncation of 50 amino acids or less compared to the corresponding naturally occurring HPV 56 L1 protein, for example 40 amino acids or less, 30 amino acids or less, 20 amino acids or less, 15 amino acids or less, 10 amino acids or less, 5 amino acids or less. In one embodiment, the HPV 56 L1 protein comprises a C-terminal truncation of 50 amino acids or less compared to SEQ ID NO: 35 or to a variant thereof, for example 40 amino acids or less, 30 amino acids or less, 20 amino acids or less, 15 amino acids or less, 10 amino acids or less, 5 amino acids or less. In one embodiment, the HPV 56 L1 protein comprises a N-terminal truncation of 50 amino acids or less compared to SEQ ID NO: 35 or to a variant thereof, for example 40 amino acids or less, 30 amino acids or less, 20 amino acids or less, 15 amino acids or less, 10 amino acids or less, 5 amino acids or less.

In one embodiment, the one or more HPV antigen(s) comprise a HPV 66 L1 protein comprising or consisting of an amino-acid sequence which is at least 80% identical to SEQ ID NO: 37, suitably at least 90%, at least 95%, 99% or 100% identical to SEQ ID NO: 37.

The sequence of the HPV 66 L1 protein may be C-terminally or N-terminally truncated compared to the corresponding naturally occurring HPV 66 L1 protein. Suitably, the HPV 66 L1 protein comprises a C-terminal truncation or N-terminal truncation of 50 amino acids or less compared to the corresponding naturally occurring HPV 66 L1 protein, for example 40 amino acids or less, 30 amino acids or less, 20 amino acids or less, 15 amino acids or less, 10 amino acids or less, 5 amino acids or less. In one embodiment, the HPV 66 L1 protein comprises a C-terminal truncation of 50 amino acids or less compared to SEQ ID NO: 37 or to a variant thereof, for example 40 amino acids or less, 30 amino acids or less, 20 amino acids or less, 15 amino acids or less, 10 amino acids or less, 5 amino acids or less. In one embodiment, the HPV 66 L1 protein comprises a N-terminal truncation of 50 amino acids or less compared to SEQ ID NO: 37 or to a variant thereof, for example 40 amino acids or less, 30 amino acids or less, 20 amino acids or less, 15 amino acids or less, 10 amino acids or less,

In one embodiment, the one or more HPV antigen(s) comprise a HPV 67 L1 protein comprising or consisting of an amino-acid sequence which is at least 80% identical to SEQ ID NO: 38, suitably at least 90%, at least 95%, 99% or 100% identical to SEQ ID NO: 38.

The sequence of the HPV 67 L1 protein may be C-terminally or N-terminally truncated compared to the corresponding naturally occurring HPV 67 L1 protein. Suitably, the HPV 67 L1 protein comprises a C-terminal truncation or N-terminal truncation of 50 amino acids or less compared to the corresponding naturally occurring HPV 67 L1 protein, for example 40 amino acids or less, 30 amino acids or less, 20 amino acids or less, 15 amino acids or less, 10 amino acids or less, 5 amino acids or less. In one embodiment, the HPV 67 L1 protein comprises a C-terminal truncation of 50 amino acids or less compared to SEQ ID NO: 38 or to a variant thereof, for example 40 amino acids or less, 30 amino acids or less, 20 amino acids or less, 15 amino acids or less, 10 amino acids or less, 5 amino acids or less. In one embodiment, the HPV 67 L1 protein comprises a N-terminal truncation of 50 amino acids or less compared to SEQ ID NO: 38 or to a variant thereof, for example 40 amino acids or less, 30 amino acids or less, 20 amino acids or less, 15 amino acids or less, 10 amino acids or less, 5 amino acids or less.

In one embodiment, the one or more HPV antigen(s) comprise a HPV 68 L1 protein comprising or consisting of an amino-acid sequence which is at least 80% identical to SEQ ID NO: 39, suitably at least 90%, at least 95%, 99% or 100% identical to SEQ ID NO: 39.

The sequence of the HPV 68 L1 protein may be C-terminally or N-terminally truncated compared to the corresponding naturally occurring HPV 68 L1 protein. Suitably, the HPV 68 L1 protein comprises a C-terminal truncation or N-terminal truncation of 50 amino acids or less compared to the corresponding naturally occurring HPV 68 L1 protein, for example 40 amino acids or less, 30 amino acids or less, 20 amino acids or less, 15 amino acids or less, 10 amino acids or less, 5 amino acids or less. In one embodiment, the HPV 68 L1 protein comprises a C-terminal truncation of 50 amino acids or less compared to SEQ ID NO: 39 or to a variant thereof, for example 40 amino acids or less, 30 amino acids or less, 20 amino acids or less, 15 amino acids or less, 10 amino acids or less, 5 amino acids or less. In one embodiment, the HPV 68 L1 protein comprises a N-terminal truncation of 50 amino acids or less compared to SEQ ID NO: 39 or to a variant thereof, for example 40 amino acids or less, 30 amino acids or less, 20 amino acids or less, 15 amino acids or less, 10 amino acids or less,

In one embodiment, the one or more HPV antigen(s) comprise a HPV 70 L1 protein comprising or consisting of an amino-acid sequence which is at least 80% identical to SEQ ID NO: 40, suitably at least 90%, at least 95%, 99% or 100% identical to SEQ ID NO: 40.

The sequence of the HPV 70 L1 protein may be C-terminally or N-terminally truncated compared to the corresponding naturally occurring HPV 70 L1 protein. Suitably, the HPV 70 L1 protein comprises a C-terminal truncation or N-terminal truncation of 50 amino acids or less compared to the corresponding naturally occurring HPV 70 L1 protein, for example 40 amino acids or less, 30 amino acids or less, 20 amino acids or less, 15 amino acids or less, 10 amino acids or less, 5 amino acids or less. In one embodiment, the HPV 70 L1 protein comprises a C-terminal truncation of 50 amino acids or less compared to SEQ ID NO: 40 or to a variant thereof, for example 40 amino acids or less, 30 amino acids or less, 20 amino acids or less, 15 amino acids or less, 10 amino acids or less, 5 amino acids or less. In one embodiment, the HPV 70 L1 protein comprises a N-terminal truncation of 50 amino acids or less compared to SEQ ID NO: 40 or to a variant thereof, for example 40 amino acids or less, 30 amino acids or less, 20 amino acids or less, 15 amino acids or less, 10 amino acids or less, 5 amino acids or less.

In one embodiment, the one or more HPV antigen(s) comprise a HPV 73 L1 protein comprising or consisting of an amino-acid sequence which is at least 80% identical to SEQ ID NO: 41, suitably at least 90%, at least 95%, 99% or 100% identical to SEQ ID NO: 41.

The sequence of the HPV 73 L1 protein may be C-terminally or N-terminally truncated compared to the corresponding naturally occurring HPV 73 L1 protein. Suitably, the HPV 73 L1 protein comprises a C-terminal truncation or N-terminal truncation of 50 amino acids or less compared to the corresponding naturally occurring HPV 73 L1 protein, for example 40 amino acids or less, 30 amino acids or less, 20 amino acids or less, 15 amino acids or less, 10 amino acids or less, 5 amino acids or less. In one embodiment, the HPV 73 L1 protein comprises a C-terminal truncation of 50 amino acids or less compared to SEQ ID NO: 41 or to a variant thereof, for example 40 amino acids or less, 30 amino acids or less, 20 amino acids or less, 15 amino acids or less, 10 amino acids or less, 5 amino acids or less. In one embodiment, the HPV 73 L1 protein comprises a N-terminal truncation of 50 amino acids or less compared to SEQ ID NO: 41 or to a variant thereof, for example 40 amino acids or less, 30 amino acids or less, 20 amino acids or less, 15 amino acids or less, 10 amino acids or less,

In one embodiment, the one or more HPV antigen(s) comprise a HPV 82 L1 protein comprising or consisting of an amino-acid sequence which is at least 80% identical to SEQ ID NO: 42, suitably at least 90%, at least 95%, 99% or 100% identical to SEQ ID NO: 42.

The sequence of the HPV 82 L1 protein may be C-terminally or N-terminally truncated compared to the corresponding naturally occurring HPV 82 L1 protein. Suitably, the HPV 82 L1 protein comprises a C-terminal truncation or N-terminal truncation of 50 amino acids or less compared to the corresponding naturally occurring HPV 82 L1 protein, for example 40 amino acids or less, 30 amino acids or less, 20 amino acids or less, 15 amino acids or less, 10 amino acids or less, 5 amino acids or less. In one embodiment, the HPV 82 L1 protein comprises a C-terminal truncation of 50 amino acids or less compared to SEQ ID NO: 42 or to a variant thereof, for example 40 amino acids or less, 30 amino acids or less, 20 amino acids or less, 15 amino acids or less, 10 amino acids or less, 5 amino acids or less. In one embodiment, the HPV 82 L1 protein comprises a N-terminal truncation of 50 amino acids or less compared to SEQ ID NO: 42 or to a variant thereof, for example 40 amino acids or less, 30 amino acids or less, 20 amino acids or less, 15 amino acids or less, 10 amino acids or less, 5 amino acids or less.

As used herein, a “variant” is a protein that differs in sequence from a reference antigen sequence but retains at least one essential property of the reference antigen. Changes in the sequence of protein variants may be limited or conservative, so that the sequences of the reference protein and the variant are closely similar overall and, in many regions, identical. A variant and reference antigen can differ in amino acid sequence by one or more substitutions, additions or deletions in any combination. A variant of an antigen can be naturally occurring such as an allelic variant, or can be a variant that is not known to occur naturally. Non-naturally occurring variants of nucleic acids and polypeptides may be made by mutagenesis techniques or by direct synthesis. In a preferred embodiment, the essential property retained by the variant is the ability to induce an immune response, suitably a humoral or Tcell response, which is similar to the immune response induced by the reference antigen. Suitably, the variant induces a humoral or Tcell response in mice which is not more than 10-fold lower, more suitably not more than 5-fold lower, not more than 2-fold lower or not lower, than the immune response induced by the reference antigen. Suitably, a HPV antigen variant has an amino acid sequence which is at least 60%, 65%, 70%, 75% 80%, 85%, 90%, 95%, 96%, 97%, 98%, 99% or 99,5% identical to the reference antigen sequence. Suitable HPV antigen variants include truncations, deletions, substitution, or insertion mutants. Suitable HPV L1 variants include truncated or mutated L1 proteins, for example truncations removing a nuclear localization signal and/or DNA binding patterns, or mutations inactivating a nuclear localization signal and/or DNA binding patterns.

In one embodiment the one or more HPV antigens are pre-adsorbed onto an aluminum metallic salt, preferably selected from aluminum oxide hydroxide (AlOOH), aluminum phosphate (AlPO₄), aluminum hydroxyphosphate sulfate (AHO₉PS⁻³) and aluminum hydroxyphosphate (AlHO₅P). In a preferred embodiment, the HPV antigens comprise HPV L1 VLPs pre-adsorbed onto an aluminum metallic salt, preferably selected from AlOOH, AlPO₄ and AlHO₉PS⁻³. In a more preferred embodiment, the HPV antigens comprise L1 VLPs from HPV types 6, 11, 16, 18, 31, 33, 45, 51 and 58, each independently pre-adsorbed onto an aluminum metallic salt, preferably selected from AlOOH, AlPO₄ and AlHO₉PS⁻³. In a more preferred embodiment, the HPV L1 VLPs from HPV types 6, 11, 16, 18, 31, 33, 45, 51 and 58 are independently pre-adsorbed onto AlOOH. The metallic salt onto which the one or more HPV antigens are pre-adsorbed is not adsorbed onto a glycolipid based TLR4 ligand.

In one embodiment, the method further comprises the step of adding a metallic salt to the HPV vaccine composition. In one embodiment, the metallic salt is added concomitantly to the addition of the non adsorbed glycolipid based TLR4 ligand in step (ii). In another embodiment, the metallic salt is added prior to the addition of the non adsorbed glycolipid based TLR4 ligand in step (ii). In another embodiment, the metallic salt is added after to the addition of the non adsorbed glycolipid based TLR4 ligand in step (ii). In a preferred embodiment, the metallic salt is added prior to the addition of the non adsorbed glycolipid based TLR4 ligand in step (ii). Suitably, the added metallic salt is selected from AlOOH, AlPO₄ and AlHO₉PS⁻³. In a preferred embodiment, the added metallic salt is AlOOH.

In one aspect, the invention provides a HPV vaccine composition obtained by the method according to the invention.

In one embodiment, the HPV vaccine composition comprises at least two, for example at least three, at least four, at least five, at least six, at least seven, at least eight, at least nine HPV antigen. In a preferred embodiment, the HPV vaccine composition comprises at least nine HPV antigens.

In one embodiment, the HPV vaccine composition comprises HPV antigen(s) from HPV types selected from HPV types 6, 11, 16, 18, 26, 31, 33, 35, 39, 45, 51, 52, 53, 56, 58, 59, 67, 68, 70, 73 and 82.

In one embodiment, the HPV vaccine composition comprises HPV antigen(s) from HPV types selected from HPV types 6, 11, 16, 18, 31, 33, 35, 39, 45, 52, 58 and 59.

In a preferred embodiment, the HPV vaccine composition comprises HPV antigen(s) from HPV types selected from HPV types 6, 11, 16, 18, 31, 33, 45, 52 and 58.

In one embodiment, the HPV vaccine composition comprises HPV antigen(s) from HPV types 16 and 18.

In one embodiment, the HPV vaccine composition comprises HPV antigen(s) from HPV types 6, 11, 16, and 18.

In one embodiment, the HPV vaccine composition comprises HPV antigen(s) from HPV types 6, 11, 16, 18, 31, 33, 45, 52 and 58.

In one embodiment, the HPV vaccine composition comprises HPV antigen(s) from HPV types 6, 11, 16, 18, 31, 33, 45, 52 and 58, and optionally HPV antigen(s) from HPV types 35, 39 and/or 59.

In one embodiment, the HPV vaccine composition comprises HPV L1 VLPs from HPV types 16 and 18.

In one embodiment, the HPV vaccine composition comprises HPV L1 VLPs from HPV types 6, 11, 16, and 18.

In one embodiment, the HPV vaccine composition comprises HPV L1 VLPs L1 VLPs from HPV types 6, 11, 16, 18, 31, 33, 45, 52 and 58 L1 VLPs, and optionally from HPV types 35, 39 and/or 59.

In one embodiment, the amount of the metallic ion which is part of the metallic salt in the HPV vaccine composition is from 100 to 500 μg/dose, suitably from 200 to 500 μg/dose, from 300 to 500 μg/dose, from 400 to 500 μg/dose or about 500 μg/dose. In a preferred embodiment, the amount of the metallic ion which is part of the metallic salt in the HPV vaccine composition is 500 μg/dose.

In one embodiment, the concentration of the metallic ion which is part of the metallic salt in the HPV vaccine composition is from 200 μg/ml to 1 mg/ml, suitably from 400 μg/ml to 1 mg/ml, from 600 μg/ml to 1 mg/ml, from 800 μg/ml to 1 mg/ml or about 1 mg/ml. In a preferred embodiment, the concentration of the metallic ion which is part of the metallic salt in the HPV vaccine composition is 1 mg/ml.

In a preferred embodiment, the metallic ion which is part of the metallic salt is Al³⁺ and the amount of Al³⁺ in the HPV vaccine composition is from 100 to 500 μg/dose, suitably from 200 to 500 μg/dose, from 300 to 500 μg/dose, from 400 to 500 μg/dose or about 500 μg/dose. In a preferred embodiment, the amount of Al³⁺ in the HPV vaccine composition is 500 μg/dose.

In a preferred embodiment, the metallic ion which is part of the metallic salt is Al³⁺ and the concentration of Al³⁺ in the HPV vaccine composition is from 200 μg/ml to 1 mg/ml, suitably from 400 μg/ml to 1 mg/ml, from 600 μg/ml to 1 mg/ml, from 800 μg/ml to 1 mg/ml or about 1 mg/ml. In a preferred embodiment, the concentration of Al³⁺ in the HPV vaccine composition is 1 mg/ml.

In one embodiment, the amount of glycolipid based TLR4 ligand in the HPV vaccine composition is from 1 to 200 μg/dose, preferably from 2 to 100 μg/dose, from 5 to 75 μg/dose, from 10 to 50 μg/dose or about 50 μg/dose. In a preferred embodiment, the amount of glycolipid based TLR4 ligand in the HPV vaccine composition is 50 μg/dose.

In one embodiment, the amount of glycolipid based TLR4 ligand in the HPV vaccine composition is from 2 to 400 μg/ml, preferably from 4 to 200 μg/ml, from 10 to 150 μg/ml, from 20 to 100 μg/ml or about 100 μg/ml. In a preferred embodiment, the amount of glycolipid based TLR4 ligand in the HPV vaccine composition is 100 μg/ml.

In a preferred embodiment, the glycolipid based TLR4 ligand is MPL, and the amount of MPL in the HPV vaccine composition is from 10 to 50 μg/dose, preferably from 20 to 50 μg/dose, from 30 to 50 μg/dose, from 40 to 50 μg/dose or about 50 μg/dose. In a preferred embodiment, the amount of MPL in the HPV vaccine composition is 50 μg/dose.

In a preferred embodiment, the glycolipid based TLR4 ligand is MPL, and the concentration of MPL in the HPV vaccine composition is from 20 to 100 μg/ml, preferably from 40 to 100 μg/ml, from 60 to 100 μg/ml, from 80 to 100 μg/ml or about 100 μg/ml. In a preferred embodiment, the concentration of MPL in the HPV vaccine composition is 100 μg/ml.

In one embodiment, the ratio of glycolipid based TLR4 ligand:metallic ion in the HPV vaccine composition is from 1:2 to 1:20, preferably from 1:3 to 1:15, more preferably from 1:5 to 1:10, for example 1:5, 1:6, 1:7, 1:8, 1:9 or 1:10.

In a preferred embodiment, the metallic ion is Al³⁺, the glycolipid based TLR4 ligand is MPL, and the ratio of MPL: Al³⁺ in the HPV vaccine composition is from 1:2 to 1:20, preferably from 1:3 to 1:15, more preferably from 1:5 to 1:10, for example 1:5, 1:6, 1:7, 1:8, 1:9 or 1:10.

Preferably, TLR4 ligand biological activity (or bioactivity) in the HPV vaccine composition of the invention is enhanced as compared to HPV vaccine composition obtained by a method where a TLR4 ligand is pre-adsorbed onto a metallic salt prior to being combined with the HPV antigens. Biological activity of a TLR4 ligand corresponds to its ability to induce the production of pro-inflammatory cytokines, such as TNF-α, and can be measured for example by using the assay described in example 1 (section entitled ‘MPL Bioassay’).

Preferably, the pH of the HPV vaccine composition is between 6.0 and 7.5, preferably between 6.5 and 7.2, for example about 6.5, about 6.6, about 6.7, about 6.8, about 6.9, about 7.0, about 7.2 or about 7.2.

The amount of the one or more HPV antigens in the HPV vaccine composition can vary with the condition, sex, age and weight of the individual, the administration route of the vaccine. The amount can also be varied with the number of HPV antigens types. Suitably the delivery is of an amount of HPV antigens suitable to generate an immunologically protective response. Suitably each vaccine dose comprises between 5 and 100 μg of each HPV antigen.

Suitably, the HPV vaccine composition can comprise pharmaceutically acceptable carriers. Suitably, the HPV vaccine composition may be prepared for administration by being suspended or dissolved in a pharmaceutically acceptable carrier. A “pharmaceutically acceptable carrier” includes any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The HPV vaccine composition may also contain a pharmaceutically acceptable diluent, such as water, saline, glycerol, etc. Additionally, auxiliary substances, such as wetting or emulsifying agents, pH buffering substances, surfactants, anti-oxidants, and the like, may be present. Sterile pyrogen-free, phosphate buffered physiologic saline is a typical carrier. The appropriate carrier may depend in large part upon the route of administration.

In one embodiment, the HPV vaccine composition comprises a surfactant, suitably a polysorbate (PS) such as PS20, PS60, PS80 or a poloxamer such as poloxamer 188. In a preferred embodiment, the surfactant comprises or consists of PS80. Suitably, the amount of the surfactant in the HPV vaccine composition is from 5 to 200 μg/dose, preferably from 10 to 100 μg/dose, more preferably from 20 to 50 μg/dose. In a preferred embodiment, the surfactant is PS80 and the amount of PS80 in the HPV vaccine composition is from 5 to 200 μg/dose, preferably from 10 to 100 μg/dose, more preferably from 20 to 50 μg/dose. Suitably, the concentration of the surfactant in the HPV vaccine composition is from 10 to 400 μg/mL, preferably from 20 to 200 μg/mL, more preferably from 40 to 100 μg/mL. In a preferred embodiment, the surfactant is PS80 and the concentration of PS80 in the HPV vaccine composition is from 10 to 400 μg/mL, preferably from 20 to 200 μg/mL, more preferably from 40 to 100 μg/m L.

In one embodiment, the HPV vaccine composition comprises a buffering agent. Suitably, the concentration of the buffering agent in the HPV vaccine composition is from 5 to 20 mM, preferably from 7,5 to 15 mM, for example 10 mM. In a preferred embodiment, the buffering agent comprises or consists of Na₂HPO₄/NaH₂PO₄. Suitably, the concentration of Na₂HPO₄/NaH₂PO₄ in the HPV vaccine composition is from 5 to 20 mM, preferably from 7,5 to 15 mM, for example 10 mM.

Suitably, the HPV vaccine composition is in liquid form, or in lyophilised form that is reconstituted into a liquid form prior to administration.

Suitably, the HPV vaccine composition is to be administered to a subject by any route as is known in the art, for example by intramuscular administration. Suitably, the subject is a vertebrate, such as a mammal, e.g. a human, a non-human primate, or a veterinary mammal (livestock or companion animals). In a preferred embodiment, the subject is a human.

The HPV vaccine composition according to the invention are useful to prevent or treat persistent HPV infection or subclinical HPV infection of the human anogenital epithelium, such as cervical epithelium, or HPV-related conditions such as CIN1 or LSIL, CIN2 and CIN3 lesions.

In one aspect, there is provided a HPV vaccine composition as described herein for use in therapy.

In one aspect, there is provided a HPV vaccine composition as described herein for preventing or treating a HPV infection or associated disease in a subject.

In one aspect, there is provided the use of a HPV vaccine composition as described herein, in the manufacture of a medicament for the prevention or treatment of HPV infection in a subject.

In one aspect, there is provided a method of preventing or treating an infection or disease comprising the administration of an effective amount of a HPV vaccine composition as described herein to a patient in need thereof.

In one aspect, there is provided a method of preventing or treating an infection or disease caused by HPV comprising the administration of an effective amount of a HPV vaccine composition as described herein to a patient in need thereof.

It will be readily understood that the immunogenic compositions of the invention are suited for use in regimens involving repeated delivery over time for therapeutic purposes.

### Sequence Comparison

For the purposes of comparing two closely-related polynucleotide or polypeptide sequences, the “sequence identity” or “% identity” between a first sequence and a second sequence may be calculated using an alignment program, such as the Basic Local Alignment Search Tool (BLAST) using standard settings. The percentage identity is the number of identical residues divided by the length of the alignment, multiplied by 100. An alternative definition of identity is the number of identical residues divided by the number of aligned residues, multiplied by 100. Alternative methods include using a gapped method in which gaps in the alignment, for example deletions in one sequence relative to the other sequence, are considered. Polypeptide or polynucleotide sequences are said to be identical to other polypeptide or polynucleotide sequences, if they share 100% sequence identity over their entire length.

A “difference” between two sequences refers to an insertion, deletion or substitution, e.g., of a single amino acid residue in a position of one sequence, compared to the other sequence.

For the purposes of comparing a first, reference polypeptide sequence to a second, comparison polypeptide sequence, the number of additions, substitutions and/or deletions made to the first sequence to produce the second sequence may be ascertained. An addition is the addition of one amino acid residue into the sequence of the first polypeptide (including addition at either terminus of the first polypeptide). A substitution is the substitution of one amino acid residue in the sequence of the first polypeptide with one different amino acid residue. A deletion is the deletion of one amino acid residue from the sequence of the first polypeptide (including deletion at either terminus of the first polypeptide).

Suitable substitutions in the sequences of the present invention may be conservative substitutions. A conservative substitution comprises the substitution of an amino acid with another amino acid having a physico-chemical property similar to the amino acid that is substituted (see, for example, Stryer et al, Biochemistry, 5th Edition 2002, pages 44-49). Preferably, the conservative substitution is a substitution selected from the group consisting of: (i) a substitution of a basic amino acid with another, different basic amino acid; (ii) a substitution of an acidic amino acid with another, different acidic amino acid; (iii) a substitution of an aromatic amino acid with another, different aromatic amino acid; (iv) a substitution of a non-polar, aliphatic amino acid with another, different non-polar, aliphatic amino acid; and (v) a substitution of a polar, uncharged amino acid with another, different polar, uncharged amino acid. A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

Terms

Unless otherwise explained in the context of this disclosure, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or ‘˜’) 200 pg.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof.

Amino acid sequences provided herein are designated by either single-letter or three-letter nomenclature, as is known in the art (see, e.g., Eur. J. Biochem. 138:9-37(1984)).

Although methods and materials similar or equivalent to those described herein can be used in the practice or testing of this disclosure, suitable methods and materials are described below.

The present invention will now be further described by means of the following non-limiting examples.

### EXAMPLES

### Example 1

### Materials and Methods

### Materials

HPV types 6, 11, 16, 18, 31, 33, 45, 52 and 58 L1 VLPs were obtained as disclosed in China patent No: ZL200610140613.0 and in Pan H, et al., 2017¹⁵, in Gu, Y. et al., 2017¹⁶, in Wang D., et al., 2017¹⁷ and in Wei M. et al. 2018^{18 .} The amino acid sequences of the HPV type 6, 11, 16, 18, 31, 33, 45, 52 and 58 L1 proteins are shown in SEQ ID NOs: 2, 4, 6, 8, 10, 12, 14, 16 and 18 respectively.
Aluminum salts:
   AlOOH (Croda), provided at a concentration of around 10 mg/mL;
   AlOOH (Ix) was prepared as follow: 1 mol/L NaOH solution was slowly added to 0.33 mol/L AlCl₃ solution with stirring at 68±2° C. The amount of NaOH was 89.2% of the weight of AlCl₃ solution, and the addition of NaOH solution was stopped when the pH reached up to 5.6±0.5. The mixture was then stirred for more than 2 hours at 68±2° C. Then, the solution was filtered with a 10±5 μm filter and distributed into 5 L blue bottle. Finally the aluminum hydroxide was autoclaved at 121 ° C. for 30 minutes, and stored at 2˜8° C.
MPL was produced internally (GSK) as a powder as described in GB2220211 A. The powder was then microfluidized in water to form a suspension, the so called MPL liquid Bulk.
HPV VLP antigen AMBs (Adsorbed Mono Bulks): each of the 9 AMBs were made by mixing an Ag working solution (V2/3) with an adjuvant working solution (V1/3) containing Alum, except for HPV-16 which was obtained by mixing Ag working solution (V2/5) with adjuvant working solution (V1/5) and was completed with water (V2/5).
   The Ag working solution was prepared from a single serotype Ag bulk, diluted with NaCl, Polysorbate 80 (PS-80) and Na₂HPO₄/NaH₂PO₄ buffer (pH 6) to a concentration of 1350 μg/mL of Ag. The phosphate concentration was 25 mM. The Ag working solution was sterile filtered on two 0.22 μm PALL Supor EKV filters with PES (Poly Ether Sulfone) membrane in series.
   The adjuvant working solution was prepared from AlOOH adjuvant bulk. The Al³⁺ concentration was diluted with NaCl and WFI (water for injection) to prepare the adjuvant working solution at a target Al³⁺ concentration of 4200 μg/mL (i.e. 155.66 mM Al³⁺). The size of the AlOOH particles in the adjuvant working solution was 21 to 34 nm, which allowed for a sterile filtration on two 0.22 μm PALL Supor EKV filters with PES membrane in series. The mixing of the Ag and adjuvant working solutions resulted in a molar ratio of 16.67 mM PO₄ to 51.89 mM Al³⁺ (or 1/3.11) in the final AMB.
   The AMBs had a target Ag concentration of 900 μg/mL for serotypes 6/11/18/31/33/45/52/58 and 540 μg/mL for HPV16. The target Al³⁺ concentration for the AMBs was 1400 μg/mL, except for HPV16 for which it is 840 μg/mL.
MPL AMB without phosphate addition: water was added, and under agitation, aluminum was added until 514.3 μg/mL Al³⁺. MPL (in liquid bulk or aggregated was added) at 321.4 μg/mL of final volume. Stirring was pursued for 30 minutes. The samples were then stored at 4° C. The MPL was aggregated by addition of NaCl 1500 mM to MPL liquid bulk to obtain a final concentration of 0.7 mg/mL MPL in 428 mM of NaCl.
MPL AMB with phosphate addition: in water, MPL liquid bulk was added up to 321.4 μg/mL of final volume. Al³⁺ was then added under agitation up to 515.3 μg/mL, followed by a 100 mM solution of Na₂HPO₄/NaH₂PO₄ at pH 5.67 up to a 6.12 mM concentration. Stirring was pursued for 30 minutes. The samples were then stored at 4° C.
9V formulations using an MPL AMB: water for injection (WFI) was added, as well as homogenized MPL AMB previously formed ad 100 μg/mL. The 9 antigen AMB were homogenized and added to the mixture under continuous agitation. Stirring was pursued for 15 minutes. The 9V drug product was stored at 4° C. until further use.
In-line formulation of 9V drug product with added Alum: the 9 antigen AMBs were homogenized in a separate flask. Alum was added ad 500 μg/dose in WFI, and the 9 antigen AMBs were added under continuous agitation, followed by MPL (liquid bulk or aggregated) ad 50 μg/dose. Stirring was pursued for 30 minutes. The obtained 9V drug product was stored at 4° C. until further use.
In-line formulation of 9V drug product without added Alum: the 9 antigen AMBs were homogenized and added to WFI under continuous agitation. MPL liquid bulk was then added ad 50 μg/dose and stirring was pursued for 30 minutes. The obtained 9V drug product was stored at 4° C. until further use.

The antigen (Ag) concentrations for each of HPV types 6/11/16/18/31/33/45/52/58 in the 9V formulations were 60/80/120/40/40/40/40/40/40 μg/mL, respectively.

### Methods

### Visual Observation

At each timepoint (one day, 7 days and 14 days after formulation), the samples were visually inspected.

### pH Measurements

In order to assess the effect of time on pH, the pH of each sample was taken just after formulation (t=0, timepoint 1), after 7days for MPL AMB and 6 days for DP (drug product) (timepoint 2) and after 14days for MPL AMB and 13 days for DP (timepoint 3).

### Size Measurement

The particle size of the 9V drug product was measured by static light scattering (SLS) over time, from the day after the 9V formulation and 2 days after the formulation for MPL AMB up to 6 weeks.

Dynamic light scattering (DLS) was also tested on aggregated MPL solution, in-line 9V DP with AlOOH (Ix) and in-line 9V DP with AlOOH (Ix) with aggregated MPL.

Samples were taken after formulation and stored in Eppendorf overnight. They were resuspended by rotation for 1 minute at 30 rpm.

### Completeness of Adsorption of HPV Antigen

The purpose of this analysis was to evaluate if the HPV antigen (Ag) present under the form of a VLP was properly adsorbed on aluminum.

Centrifugation of the sample (16.000 g. 15 minutes) was performed in order to provide the alum and the adsorbed antigen as sediments. Subsequently the protein content in the supernatant (SN) was measured by μBCA. In μBCA the protein content was quantified in an indirect manner by measuring the colorimetric changes induced by the chelation of BCA (bicinchoninic acid) with reduced Cu⁺ ions in the presence of protein.

### MPL Bioassay

The biological activity of MPL was tested by assessing its ability to induce pro-inflammatory cytokine production (i.e. TNF-α) by the human monocytic cell line U937. In this assay, the cell line was differentiated into macrophage in presence of PMA and stimulated to express the TLR4 receptor which binds to MPL and cytokines secretion via TLR-4 pathway. In the presence of MPL this receptor initiates an intracellular cascade leading to the production of TNF-α. The TNF-α binds to beads coated with TNF-a specific Ab. A secondary Ab coated with a fluorophore recognizes the bound TNF-α. A FACS system is used to count and characterize the TNF-α containing beads. This signal can be linked back to the TNF-α concentration.

The production of TNF-α was measured in the supernatants with CBA (Cytometric Bead Array) Flex kits (Becton Dickinson) leading to absolute values of cytokine production in pg/ml. The data generated were expressed as Relative Potency (RP) by performing ratio between each measure (replicate) of TNFα cytokine secretion after stimulation with MPL based formulations at 3 concentrations (1, 3 and 10 μg/mL) and the arithmetic mean value of the quadruplicates of the reference MPL lot.

### MPL Adsorption Assessment

The purpose of this analysis was to quantify the MPL content in the supernatant of the formulation after one day. The sample was first centrifuged at 16 000 g for 15 minutes to ensure that all aluminum is pelleted. The supernatant was then recovered and analyzed by RP-HPLC fluo. The detected MPL allowed determination of the completeness of MPL adsorption.

### Example 2

### List of Tested Formulations

The following formulations were tested in two sets of experiments.

### Experiment A

| | |
|---|---|
| Formulation | Sample name |

9V DP in-line formulation

| | |
|---|---|
| In-line AlOOH (lx) added | 20HPC03001 |
| In-line no Al added | 20HPC03002 |
| In-line AlPO₄ added | 20HPC03003 |
| In-line AlOOH (Croda) added | 20HPC03004 |

9V DP in-line formulation with aggregated MPL

| | |
|---|---|
| In-line AlOOH (lx) added + aggregated MPL | 20HPC03005 |
| In-line AlPO₄ added + aggregated MPL | 20HPC03006 |
| In-line AlOOH (Croda) added + aggregated MPL | 20HPC03007 |

9V DP formulation with AMB MPL (not in-line)

| | |
|---|---|
| 9V DP with AMB MPL AlPO₄ | 20HPC03008 |
| 9V DP with AMB MPL AlOOH (Croda) | 20HPC03009 |
| 9V DP with AMB aggregated MPL AlPO₄ | 20HPC03010 |

DP: Drug product

### Experiment B

| Formulation | Sample name |
|---|---|
| 9V DP with MPL adsorbed on AlOOH (lx) with PO₄ | 20HPC01007 |
| at RT | 20HPC02007 |
| 9V DP with MPL adsorbed on AlPO₄ at RT | 20HPC01037 |
| | 20HPC02027 |
| 9V DP with MPL adsorbed on AlOOH (lx) without PO₄ | 20HPC01017 |
| at RT | 20HPC02043 |
| 9V DP with MPL added ‘in-line’ AlOOH (lx) added | 20HPC010272 |
| | 0HPC02051 |
| 9V DP without MPL (420 μg Al³⁺/dose) | 20HPC02061 |
| Cervarlx-like control (100 μg MPL on 500 μg ALOOH/ | 20HPC02071 |
| dose) | |
| 9V placebo | 20HPC02082 |
| Dilution buffer | 20HPC02083 |

### Example 3

### Visual Observation

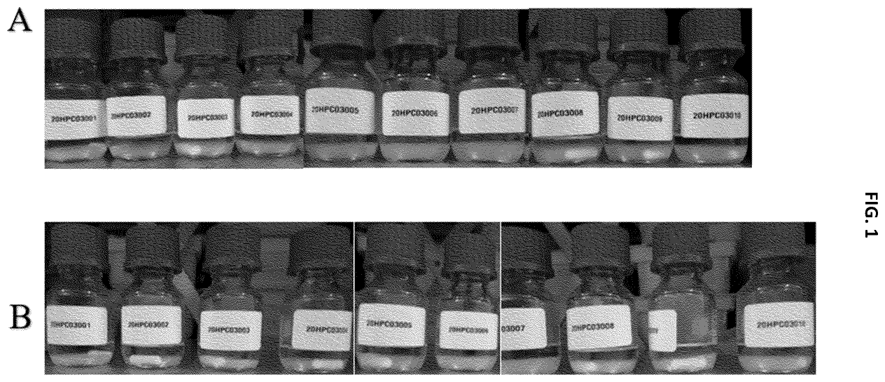

Experiment A—No major change was observed between the various formulations at the two time points (FIG. 1). The aluminum layer was thinner after 7 days and no flocculation was observed.

Experiment B—All samples were visually inspected and were all found to be opalescent.

### Example 4

### pH Measurements

Experiment A—Measured pH are summarized in Table 2. The pH of the samples did not evolve over time. In a range of 0.5 pH units, the pH was similar for each drug product formulation.

TABLE 2
pH measured for every sample at each timepoint. 1 is
t = 0 (after formulation); 2 is 6 days; 3 is 13 days.

| | | pH | pH | pH |
|---|---|---|---|---|
| | | at | at | at |
| Formulation | Sample name | 1 | 2 | 3 |
| 9V DP In-line AlOOH (lx) added | 20HPC03001 | 6.86 | 6.91 | 6.87 |
| 9V DP In-line no Al added | 20HPC03002 | 7.25 | 7.25 | 7.21 |
| 9V DP In-line AlPO₄ added | 20HPC03003 | 6.86 | 6.94 | 6.97 |
| 9V DP In-line AlOOH (Croda) | 20HPC03004 | 7.16 | 7.19 | 7.16 |
| added | | | | |
| 9V DP In-line AlOOH (lx) added + | 20HPC03005 | 6.85 | 6.86 | 6.89 |
| aggregated MPL | | | | |
| 9V DP In-line AlPO₄ added + | 20HPC03006 | 6.9 | 6.96 | 6.94 |
| aggregated MPL | | | | |
| 9V DP In-line AlOOH (Croda) | 20HPC03007 | 7.14 | 7.1 | 7.15 |
| added + aggregated MPL | | | | |
| 9V DP with MPL AMB AlPO₄ | 20HPC03008 | 7.08 | 7.07 | 7.1 |
| 9V DP with MPL AMB AlOOH | 20HPC03009 | 7.16 | 7.25 | 7.18 |
| (Croda) | | | | |
| 9V DP with aggregated MPL AMB | 20HPC03010 | 7.06 | 7.1 | 7.08 |
| AlPO₄ | | | | |

Experiment B—The pH was measured three hours after the formulation of the 9V DP, the resulting data is presented in table 3. The long-term storage pH of the HPV AMBs was about 6.7 (data not shown). It is to be noted that the stability of the VLPs is ensured at this pH.

TABLE 3
pH measured for the 9V drug products 3 hours after formulation.

| Formulation | Sample name | pH |
|---|---|---|
| 9V based on MPL AMB + AlOOH (lx) + PO₄ | 20HPC02007 | 6.85 |
| 9V based on MPL AMB + AlPO₄ | 20HPC02027 | 6.94 |
| 9V based on MPL AMB + AlOOH (lx) | 20HPC02043 | 6.76 |
| MPL in-line (AlOOH lx) | 20HPC02051 | 6.78 |
| 9V without MPL & 420 μg Al/dose | 20HPC02061 | 7.07 |

### Example 5

### Size Measurements

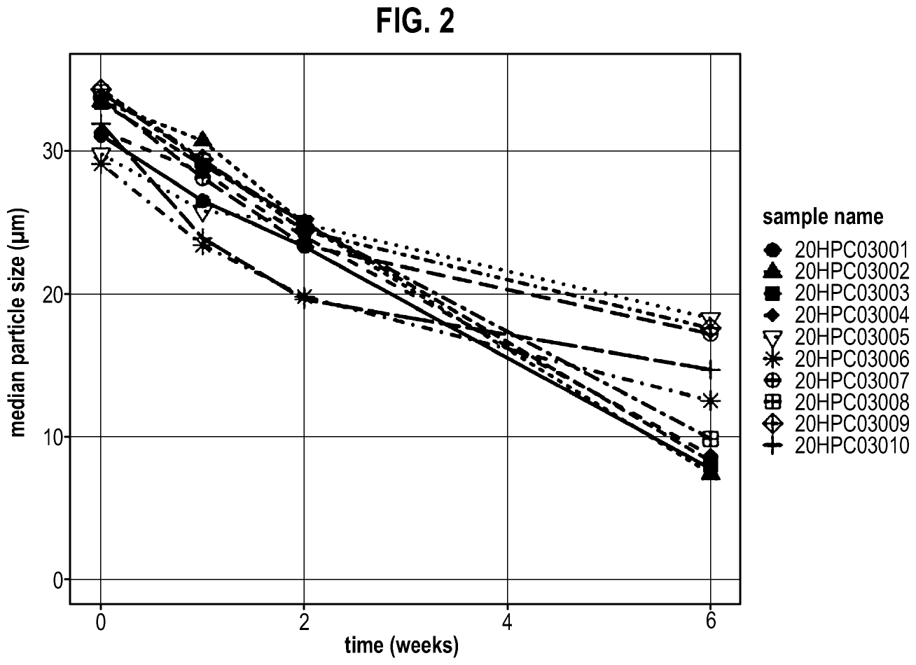

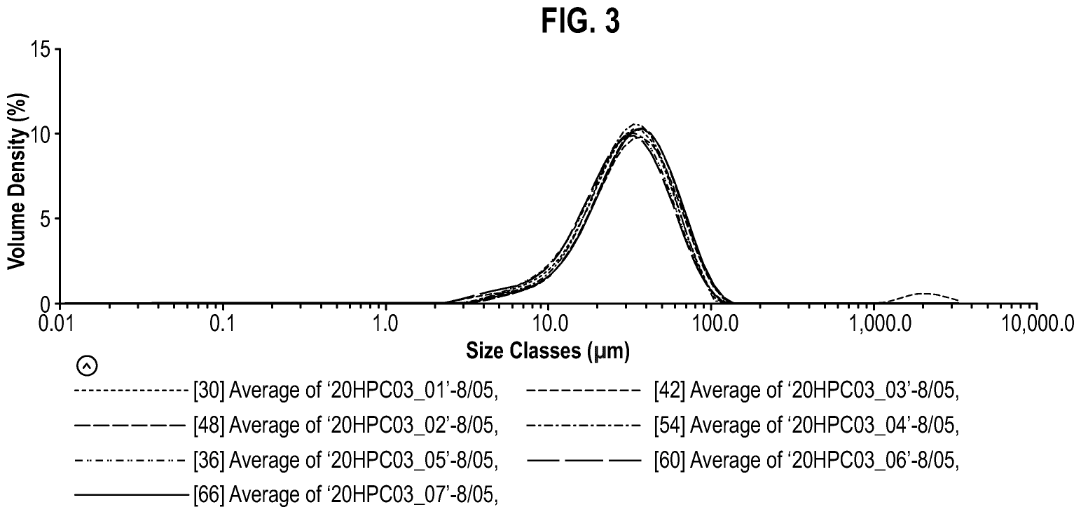

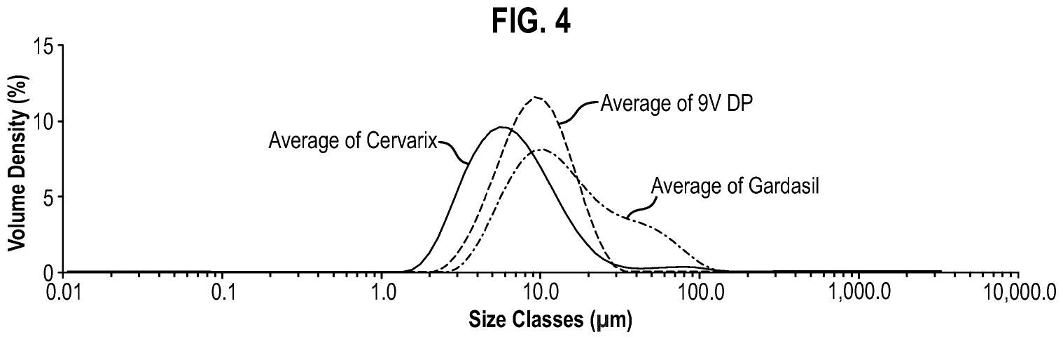

Experiment A—The average median particle size of 5 consecutive measurements are given in table 4 and in FIG. 2. Size distribution is shown for all 9V DPs (with or w/o aggregated MPL) in FIG. 2 and FIG. 3. FIG. 4 shows size distribution for CERVARIX, GARDASIL-9 and a 9V DP composed by the 9 serotypes in the matrix without addition of MPL. DLS analysis were also performed on in-line 9V DP with AlOOH (Ix) and in-line 9V DP with AlOOH (Ix) with aggregated MPL. No difference was observed between aggregated and non-aggregated MPL.

FIG. 2 highlights the decrease of the size of the particles over 4 time points (1 day after formulation, one week, two weeks and six weeks), and FIG. 3 shows that all 9V DP formulations have a nearly identical monomodal peak with a median between 29 to 32 μm depending on the formulation. FIG. 4 shows that the 3 reference DP have a similar size which is smaller in comparison with the tested 9V DP formulations. The 9V DP without MPL was also found to have 2 distinct populations when the 2 commercial DP were mainly composed by one population.

TABLE 4
Static Light Scattering measurements performed on all samples.
The median of the peak shown are the average of 5 consecutive
measurements. The measurement was done with a new sample at
3 different timepoints. 1 is t = 0 (after formulation);
2 is 6 days; 3 is 13 days; 4 is 6 weeks after formulation.

| | | Dx | Dx | Dx | Dx |
|---|---|---|---|---|---|
| | | (50) | (50) | (50) | (50) |
| Formulation | Sample name | at 1 | at 2 | at 3 | at 4 |
| 9V DP In-line AlOOH (lx) | 20HPC03001 | 31.1 | 26.5 | 23.3 | 7.74 |
| added | | | | | |
| 9V DP In-line no Al added | 20HPC03002 | 33.5 | 30.7 | 24.9 | 7.39 |
| 9V DP In-line AlPO₄ added | 20HPC03003 | 33.4 | 29 | 25 | 8.27 |
| 9V DP In-line AlOOH | 20HPC03004 | 31.4 | 28.5 | 24 | 8.7 |
| (Croda) added | | | | | |
| 9V DP In-line AlOOH (lx) | 20HPC03005 | 29.8 | 25.8 | 24.9 | 18.3 |
| added + aggregated MPL | | | | | |
| 9V DP In-line AlPO₄ | 20HPC03006 | 29.1 | 23.4 | 19.8 | 12.5 |
| added + aggregated MPL | | | | | |
| 9V DP In-line AlOOH | 20HPC03007 | 33.7 | 28.1 | 23.4 | 17.2 |
| (Croda) added + | | | | | |
| aggregated MPL | | | | | |
| 9V DP with MPL AMB | 20HPC03008 | 34.1 | 29.3 | 24.9 | 9.83 |
| AlPO₄ | | | | | |
| 9V DP with MPL AMB | 20HPC03009 | 34.3 | 29.4 | 24.4 | 17.6 |
| AlOOH (Croda) | | | | | |
| 9V DP with aggregated | 20HPC03010 | 31.9 | 23.9 | 19.6 | 14.7 |
| MPL AMB AlPO₄ | | | | | |
| CERVARLX | / | 6.28 | / | / | / |
| GARDASIL | | 9.13 | / | / | / |
| 9V DP provided by | / | 13.1 | / | / | / |
| Innovax | | | | | |

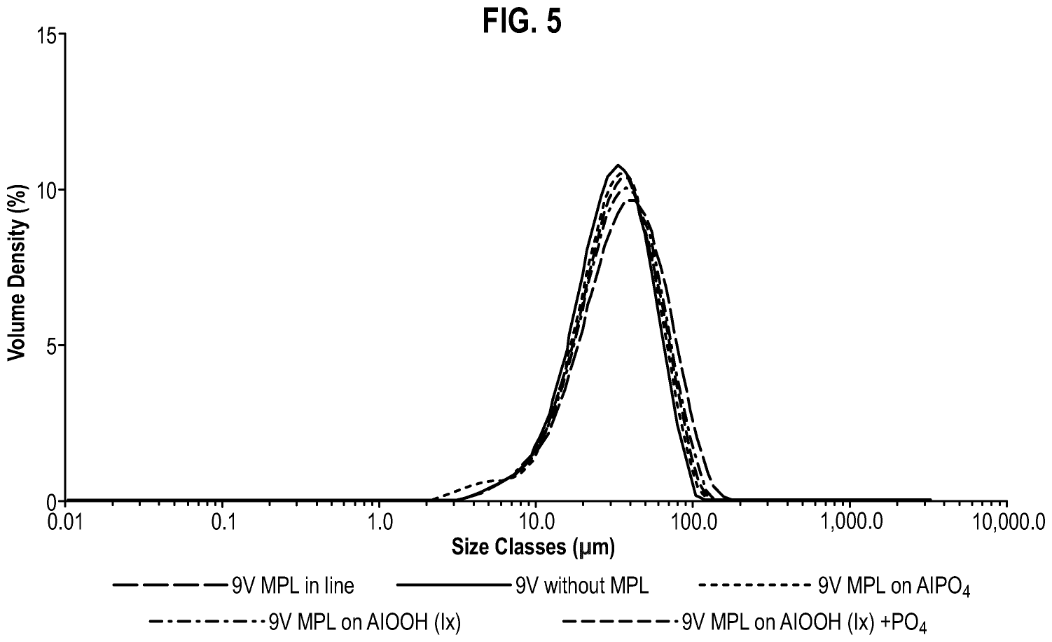

Experiment B—SLS measurements of the different formulations of the 9V DPs provided similar results for all formulations in particle size and distribution, as illustrated in table 5 and FIG. 5. The addition of MPL slightly increased the overall size of the particles as shown by the values in the last two columns of table 5. On the other hand, the 9V DP with MPL adsorbed on AlPO₄ showed a slight tail towards smaller particle sizes.

TABLE 5
Particle size measurements of the different 9V DP formulations
by SLS. The average result of 5 measurements are shown.

| | | Percentiles on | | |
|---|---|---|---|---|
| | | particle diameter (μm) | | |
| | | Dx | Dx | Dx |
| Formulation | Sample name | (10%) | (50%) | (90%) |
| 9V based on MPL AMB + | 20HPC02007 | 14.2 | 33.2 | 65.3 |
| AlOOH (lx) + PO₄ | | | | |
| 9V based on MPL AMB + | 20HPC02027 | 13.6 | 32.3 | 63.3 |
| AlPO₄ | | | | |
| 9V based on MPL AMB + | 20HPC02043 | 14.2 | 34 | 68.7 |
| AlOOH (lx) | | | | |
| MPL in-line (AlOOH lx) | 20HPC02051 | 14.7 | 36.5 | 75.5 |
| 9V without MPL & 420 μg | 20HPC02061 | 13.8 | 31.1 | 59.9 |
| Al/dose | | | | |

### Example 6

### Completeness of Adsorption of HPV Antigen

Experiment A—The quantity of total Ag which was not adsorbed on aluminum is disclosed in table 6 which reveals that all formulations adsorbed nearly all HPV antigen.

TABLE 6
Amount of HPV antigen detected in the supernatant
obtained by μBCA. This value was used to determine
the completeness of adsorption in % showed in the
third column. Limit of quantification is 6 μg/mL.

| | | Protein | Adsorption |
|---|---|---|---|
| | | in SN | completeness |
| Sample | Sample name | (μg/mL) | (%) |
| 9V DP In-line AlOOH (lx) | 20HPC03001 | 4.2 | >99 |
| added | | | |
| 9V DP In-line no Al added | 20HPC03002 | 4.3 | >99 |
| 9V DP In-line AlPO₄ added | 20HPC03003 | 4.3 | >99 |
| 9V DP In-line AlOOH (Croda) | 20HPC03004 | 4.9 | >99 |
| added | | | |
| 9V DP In-line AlOOH (lx) | 20HPC03005 | 7.7 | 98.6 |
| added + aggregated MPL | | | |
| 9V DP In-line AlPO₄ (Croda) | 20HPC03006 | 7.5 | 98.6 |
| added + aggregated MPL | | | |
| 9V DP In-line AlOOH (Croda) | 20HPC03007 | 8.5 | 98.4 |
| added + aggregated MPL | | | |
| 9V DP with MPL AMB AlPO₄ | 20HPC03008 | 3.7 | >99 |
| 9V DP with MPL AMB AlOOH | 20HPC03009 | 3.3 | >99 |
| (Croda) | | | |
| 9V DP with aggregated MPL | 20HPC03010 | 7.5 | 98.6 |
| AMB AlPO₄ | | | |

Experiment B—The results are presented in table 7. As 540 μg of HPV protein was present per 1 mL of 9V drug product, only around 1% of protein was not adsorbed and found in the supernatant of the formulation. It was remarkable that for the formulation without MPL, the adsorption was the lowest with ≥2% of HPV antigen not adsorbed.

TABLE 7
Results of the μBCA assay, quantifying the amount of non adsorbed
HPV antigen that was present in the supernatant of the different
9V drug products after centrifugation. *The LoQ is 6 μg/mL.

| | | Protein | Adsorption |
|---|---|---|---|
| | | in SN | completeness |
| Formulation | Sample name | (μg/mL) | (%) |
| 9V based on MPL AMB + | 20HPC02007 | 7.2 | 98.7 |
| AlOOH (lx) + PO₄ | | | |
| 9V based on MPLAMB + | 20HPC02027 | 4.2* | >98.9 |
| AlPO₄ | | | |
| 9V based on MPL AMB + | 20HPC02043 | 6.6 | 98.7 |
| AlOOH (lx) | | | |
| MPL in-line (AlOOH lx) | 20HPC02051 | 7.1 | 98.7 |
| 9V without MPL & 420 μg | 20HPC02061 | 11.4 | 97.9 |
| Al/dose | | | |

### Example 7

### MPL Activity Assessment

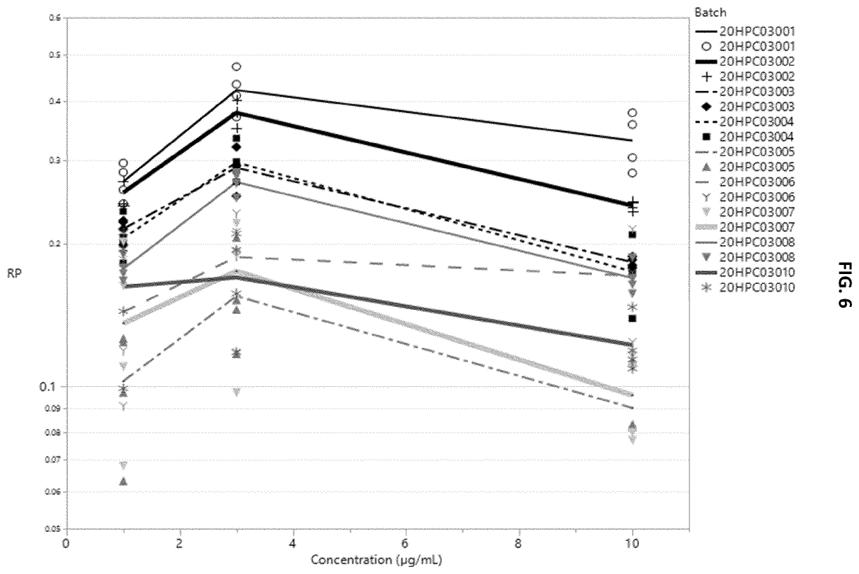

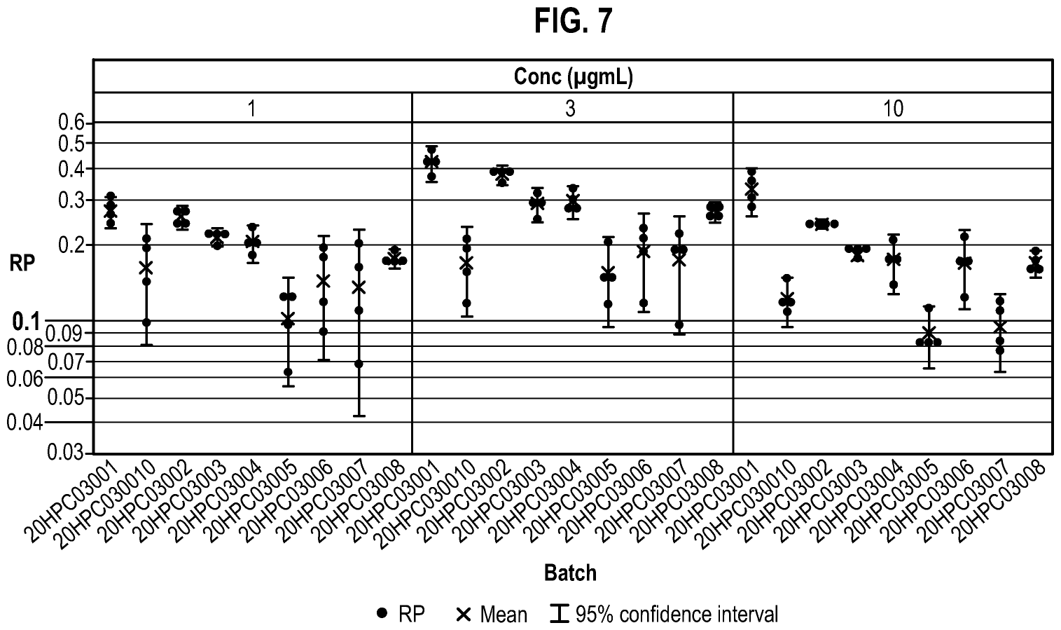

Experiment A—The results are summarized in table 8. Those values are shown in a graph in FIG. 6 and FIG. 7.

TABLE 8
Relative potency and its CV (coefficient of variation) computed with MPL ref
10 μg/mL for each sample at each concentration. (1, 3, 10 μg of MPL/mL)

| | 9 V DP in-line | | | | |
|---|---|---|---|---|---|
| | | | 20HPC03004 | | 20HPC03006 |
| | 20HPC03001 | 20HPC03002 | AlOOH | 20HPC03003 | Aggregated MPL with |
| | AlOOH | No Al | (Croda) | AlPO₄ | AlPO₄ |
| | (Ix) added | added | added | added | added |
| N | 4 | 4 | 4 | 4 | 4 |
| 1 | Mean | 0.27 | 0.26 | 0.21 | 0.22 |
| μg/mL | CV(%) | 8.73 | 6.54 | 10.49 | 5.28 |
| 3 | Mean | 0.42 | 0.38 | 0.30 | 0.29 |
| μg/mL | CV(%) | 10.21 | 5.63 | 9.16 | 9.69 |
| 10 | Mean | 0.33 | 0.24 | 0.17 | 0.18 |
| μg/mL | CV(%) | 13.56 | 2.23 | 16.40 | 2.50 |

| | 9 V DP in-line | | 9 V DP with MPL AMB | | | |
|---|---|---|---|---|---|---|
| | 20HPC03007 | 20HPC03005 | | | 20HPC03008 | 20HPC030010 |
| | Aggregated MPL with | | | | 9 V with | 9 V with |
| | AlOOH | AlOOH | | | MPL AMB | aggregated |
| | (Croda) | (Ix) | Cervarix | | on | MPL on AMB |
| | added | added | (not in-line) | | AlPO₄ | with AlPO₄ |
| N | 4 | 4 | 4 | | 4 | 4 |
| 1 | 0.14 | 0.14 | 0.10 | 0.14 | 0.18 | 0.16 |
| μg/mL | 32.03 | 43.34 | 28.75 | 10.17 | 5.53 | 31.59 |
| 3 | 0.19 | 0.18 | 0.16 | 0.24 | 0.27 | 0.17 |
| μg/mL | 26.51 | 30.80 | 24.01 | 13.54 | 5.36 | 24.17 |
| 10 | 0.17 | 0.10 | 0.09 | 0.38 | 0.17 | 0.12 |
| μg/mL | 21.76 | 21.00 | 17.05 | 48.37 | 7.63 | 13.93 |

FIGS. 6 and 7 show the relative potency (RP) of the various samples that were prepared. Formulations made with aggregated MPL had a lower RP than formulations with non aggregated MPL (liquid bulk).

The in-line formulation with Al (Ix) had the highest RP followed by the in-line with no addition of aluminum suggesting a higher in vitro bioactivity. The other in-line formulations with non aggregated MPL showed comparable relative potency.

The formulation based on MPL AMB showed the lowest response compared to the in-line formulation.

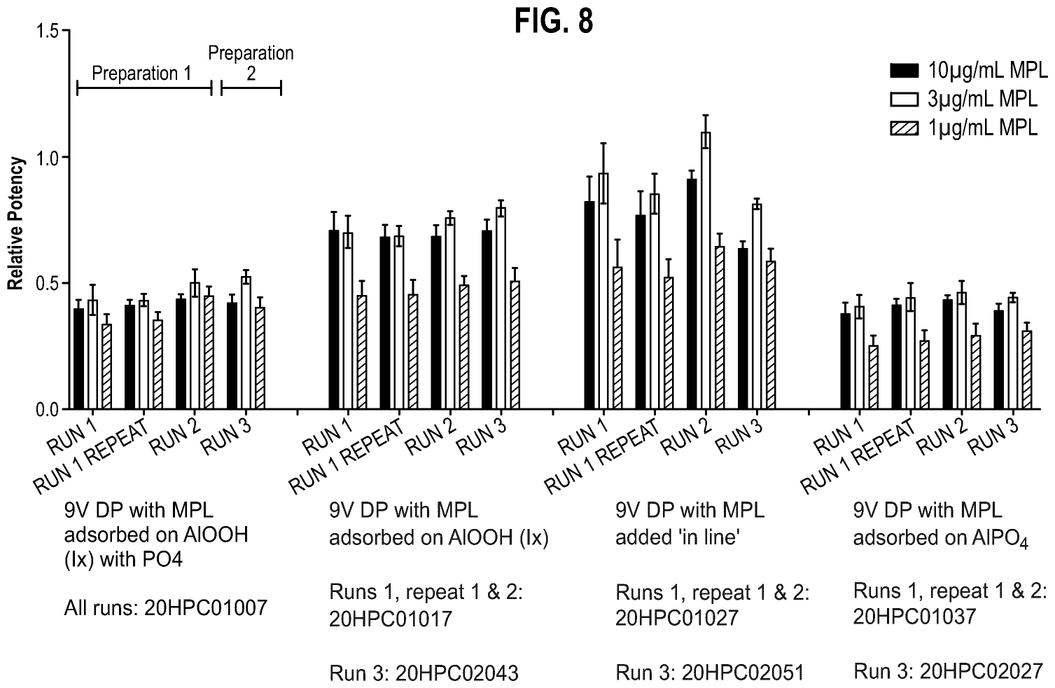

Experiment B—A statistical analysis of the relative potencies for the 9V DP formulations is shown in FIG. 8. The 9V DP with the in-line formulation provided the best relative potencies at the three concentrations in comparison to the 9V DP obtained with MPL AMB. The response was dependent on the concentration of MPL that was used (10 μg/mL vs 3 μg/mL vs 1 μg/mL), whatever the formulation.

### Example 8

### Completeness of Adsorption of MPL

Experiment A—All in-line formulations showed a complete adsorption of MPL as highlighted in table 9. The MPL AMB AlPO 4 adsorption was incomplete.

TABLE 9
MPL content in supernatant quantified by RP-HPLC-FLUO.
The completeness of adsorption of MPL on aluminium
was determined with this value. The values indicate
the concentration of MPL (μg/mL) present in the
supernatant. <25 μg/mL means that no peak was detected.

| | | MPL | Adsorption |
|---|---|---|---|
| | | in SN | completeness |
| Formulation | Sample name | (μg/mL) | (%) |
| 9V DP In-line AlOOH (lx) | 20HPC03001 | <25 | >75 |
| added | | | |
| 9V DP In-line no Al added | 20HPC03002 | <25 | >75 |
| 9V DP In-line AlPO₄ added | 20HPC03003 | <25 | >75 |
| 9V DP In-line AlOOH (Croda) | 20HPC03004 | <25 | >75 |
| 9V DP | | | |
| 9V DP with MPL AMB AlPO₄ | 20HPC03008 | <25 | >75 |
| 9V DP with MPL AMB AlOOH | 20HPC03009 | <25 | >75 |
| (Corda) | | | |

Experiment B—As illustrated in table 10, no free MPL was detected in the supernatant of the 9V DPs using the RP-HPLC-FLR method whether they were obtained with an in-line formulation or with MPL AMB.

TABLE 10
Completeness of adsorption of MPL on Alum in the 9V
DP formulations, measured by the RP-HPLC-FLR (fluo)
method (LoQ: 25 μg/mL). The values indicate the
concentration of MPL (μg/mL) present in the supernatant.

| | | MPL | Adsorption |
|---|---|---|---|
| | | in SN | completeness |
| Formulation | Sample name | (μg/mL) | (%) |
| 9V based on MPL | 20HPC02007 | <25 | >75 |
| AMB + AlOOH (lx) + | | | |
| PO4 | | | |
| 9V based on MPL | 20HPC02027 | <25 | >75 |
| AMB + AlPO₄ | | | |
| 9V based on MPL | 20HPC02043 | <25 | >75 |
| AMB + AlOOH (lx) | | | |
| MPL in-line | 20HPC02051 | <25 | >75 |
| (AlOOH lx) | | | |
| 9V without MPL & | 20HPC02061 | ND | ND |
| 420 μg Al/dose | | | |

### REFERENCES

1. R.J. Martinez-Portilla, M. Rial-Crestelo, M.A. Mejia-Ugarte, J.L. Lopez-Velazquez, Risk of cervical cancer and squamous intraepithelial lesions according to human papillomavirus high-risk serotypes detected by qualitative real-time in vitro PCR, European journal of gynaecological oncology, XL, n. 1, 2019.

2. Arbyn, M., Tommasino, M., Depuydt, C., & Dillner, J. (2014). Are 20 human papillomavirus types causing cervical cancer?. The Journal of pathology, 234(4), 431-435.

3. Bosch, F. X., Broker, T. R., Forman, D., Moscicki, A. B., Gillison, M. L., Doorbar, J., ... & Cuzick, J. (2013). Comprehensive control of human papillomavirus infections and related diseases. Vaccine, 31, H1-H31.

4. C.B. Fox, D. Carter, R.M. Kramer, A.M. Beckman, S.G. Reed, Current Status of Toll-Like Receptor 4 Ligand Vaccine Adjuvants, 2017, Chapter 6, Immunopotentiators in Modern Vaccines.

5. S.G. Reed, F Hsu, D. Carter, M. T Orr, The Science of Vaccine Adjuvants: Advances in TLR4 Ligand Adjuvants, Current Opinion in Immunology, 2016, 41: 85-90

6. Ji Eun Han, Seo Ri Wui, Kwang Sung Kim, YangJe Cho, Wan Je Cho, Na Gyong Lee, Characterization of the Structure and Immunostimulatory Activity of a Vaccine Adjuvant, De-O-Acylated Lipooligosaccharide, PLOS ONE, January 2014, Volume 9, Issue 1, e85838

7. Chou, Yi-Ju, et al., Vaccine adjuvant activity of a TLR4-activating synthetic glycolipid by promoting autophagy, Scientific Reports 10.1 (2020): 1-15.

8. Atsushi Shimoyama and Koichi Fukase, Chemically Synthesized TLR4 Ligands, Their Immunological Functions, and Potential as Vaccine Adjuvant, 2020, In: Rossetti C., Peri F. (eds) The Role of Toll-Like Receptor 4 in Infectious and Non Infectious Inflammation. Progress in Inflammation Research, vol 87. Springer, Cham.

9. Cartesio D'Agostini, Francesca Pica, Giuseppe Febbraro, Sandro Grelli, Carlo Chiavarolib, Enrico Garacia, Antitumour effect of OM-174 and Cyclophosphamide on murine B16 melanoma in different experimental conditions, International Immunopharmacology 5 (2005) 1205-1212.

10. Samar Hamdy, Azita Haddadi, Vishwa Somayaji, David Ruan, John Samuel, Pharmaceutical analysis of synthetic lipid A-based vaccine adjuvants in poly (d,l-lactic-co-glycolic acid) nanoparticle formulations, Journal of Pharmaceutical and Biomedical Analysis 44 (2007) 914-923

11. Kuramitsu, Yasuhiro, Nishibe, Manabu, Ohiro, Youichi, et al. A new synthetic lipid A analog, ONO-4007, stimulates the production of tumor necrosis factor-[alpha] in tumor tissues, resulting in the rejection of transplanted rat hepatoma cells. Anticancer Drugs. 1997;8(5):500-508.

12. Naupu, P. N., van Zyl, A. R., Rybicki, E. P., & Hitzeroth, I. I. (2020). Immunogenicity of Plant-Produced Human Papillomavirus (HPV) Virus-Like Particles (VLPs). Vaccines, 8(4), 740.

13. Kawther K. Ahmed, Sean M. Geary, and Aliasger K. Salem, Development and Evaluation of Biodegradable Particles Co-loaded with Antigen and the Toll-like Receptor Agonist, Pentaerythritol Lipid A, as a Cancer Vaccine, J Pharm Sci. 2016 March; 105(3): 1173-1179.

14. Seo Ri Wui, Hye Kyeong Kim, Ji Eun Han, Jong Min Kim, Yeon Hee Kim, Jeong Hoon Chun, Yang Je Cho, Na Gyong Lee, A combination of the TLR4 agonist CIA05 and alum promotes the immune responses to Bacillus anthracis protective antigen in mice, International Immunopharmacology 11 (2011) 1195-1204

15. Pan H#, Li Z#, Wang J, Song S, Wang D, Wei M, Gu Y, Zhang J, Li S*, Xia N*. (2017) Bacterially expressed human papillomavirus type 6 and 11 bivalent vaccine: Characterization, antigenicity and immunogenicity. Vaccine 35, 3222-3231.

16. Gu, Y.#, Wei, M.#, Wang, D., Li, Z., Xie, M., Pan, H., Wu, T., Zhang, J., Li, S*., and Xia, N*. (2017) Characterization of an Escherichia coli-derived human papillomavirus type 16 and 18 bivalent vaccine. Vaccine 35, 4637-4645.

17. Wang D#, Fan F#, Li Z, Liu X, Song S, Wei S, He M, Lin Y, Li Z, Wei M, Yu H, Gu Y, Li S*, Xia N, Stop codon mutagenesis for homogenous expression of human papillomavirus L1 protein in Escherichia coli[J]. Protein Expr Purif, 2017. 133: 110-120.

18. Wei M#, Wang D#, Li Z#, Song S, Kong X, Mo X, Yang Y, He M, Li Z, Huang B, Lin Z, Pan H, Zheng Q, Yu H, Gu Y, ZhangJ, Li S*, Xia N*. N-terminal truncations on L1 proteins of human papillomaviruses promote their soluble expression in Escherichia coli and self-assembly in vitro. Emerging Microbes & Infections. 2018 Sep 26;7(1):160.

1. A method for the preparation of a human papillomavirus (HPV) vaccine composition comprising the following steps:
(i) adsorption of one or more HPV antigen(s) on a metallic salt, and
(ii) addition of a non adsorbed glycolipid based TLR4 ligand to the mixture obtained in

2. The method according to claim 1, wherein the one or more HPV antigen(s) are selected from the group consisting of HPV late proteins L1, L2, chimeric L1 and chimeric L1/L2 proteins.

3. The method according to claim 2, wherein the one or more HPV antigen(s) are selected from HPV L1 VLPs (virus like particles).

4. The method according to claim 3, wherein the HPV L1 VLPs are produced in E. coli, yeast cells or in a baculovirus expression system.

5. The method according to claim 4, wherein the HPV L1 VLPs are produced in E. coli.

6. The method according to claim 1, wherein each of the one or more HPV antigen(s) are from a HPV type selected from HPV types 6, 11, 16, 18, 26, 31, 33, 35, 39, 45, 51, 52, 53, 56, 58, 59, 67, 68, 70, 73 and 82, more preferably from HPV types 6, 11, 16, 18, 31, 33, 115, 52 and 58.

7. The method according to claim 1, wherein the one or more HPV antigen(s) comprise HPV antigen(s) from HPV types 16 and 18.

8. The method according to claim 1, wherein the one or more HPV antigen(s) comprise HPV antigen(s) from HPV types 6, 11, 16, and 18.

9. The method according to claim 1, wherein the one or more HPV antigen(s) comprise HPV antigen(s) from HPV types 6, 11, 16, 18, 31, 33, 45, 52 and 58.

10. The method according to claim 9, wherein the one or more HPV antigen(s) further comprise HPV antigen(s) from HPV types 35, 39 and/or 59.

11. The method according to claim 1, wherein the one or more HPV antigen(s) comprise L1 VLPs from HPV types 16 and 18.

12. The method according to claim 1, wherein the one or more HPV antigen(s) comprise L1 VLPs from HPV types 6, 11, 16, and 18.

13. The method according to claim 1, wherein the one or more HPV antigen(s) comprise or consist of L1 VLPs from HPV types 6, 11, 16, 18, 31, 33, 45, 52 and 58.

14. The method according to claim 13, wherein the one or more HPV antigen(s) further comprise L1 VLPs from HPV types 35, 39 and/or 59 L1 VLPs.

15. The method according to claim 1, wherein the metallic salt comprises aluminum.

16. The method according to claim 15, wherein the metallic salt is selected from the group consisting of AlOOH, AlPO₄, AlHO₉PS⁻³ and AlHO₅P.

17. The method according to claim 1, wherein the glycolipid based TLR4 agonist is selected from MPL, GLA, SLA, OM-174, CCL-34, RC-529, PET-Lipid A, ONO-4007 and dLOS.

18. The method according to claim 1 further comprising the step of adding a metallic salt to the HPV vaccine composition.

19. The method of claim 1, wherein
the one or more HPV antigen(s) comprise L1 VLPs from HPV types 6, 11, 16, 18, 31, 33, 45, 52, and 58,
the metallic salt comprises aluminum, and
the glycolipid based TLR4 agonist is MPL.

20. A HPV vaccine composition obtained by the method of claim 1.

21. The HPV vaccine composition of claim 20 wherein the metallic ion which is part of the metallic salt is Al⁺ and the amount of Al⁺ in the HPV vaccine composition is from 100 to 500 μg/dose.

22. The HPV vaccine composition of claim 20 wherein the glycolipid based TLR4 ligand is MPL and the amount of MPL in the HPV vaccine composition is from 10 to 50 μg/dose.

23. The HPV vaccine composition of claim 20, wherein the ratio of glycolipid based TLR4 ligand:metallic salt in the HPV vaccine composition is from 1:2 to 1:20.

24. (canceled)

25. The HPV vaccine of claim 20 for preventing or treating a HPV infection or associated disease in a subject.

26. A method of preventing or treating an infection or disease caused by HPV comprising the administration of an effective amount of the HPV vaccine of to claim 20 to a patient in need thereof.

27. The method according to claim 1, wherein the one or more HPV antigen(s) consist of L1 VLPs from HPV types 6, 11, 16, 18, 31, 33, 45, 52 and 58.
